(19) United States
(12) Patent Application Publication
(10) Pub. No.: US 2022/0281999 A1
(43) Pub. Date: Sep. 8, 2022

(54) SEPARATED PEPTIDE

(71) Applicant: National University Corporation Tokai National Higher Education and Research System, Nagoya-shi, Aichi (JP)

(72) Inventor: Katsutoshi Hori, Nagoya-shi, Aichi (JP)

(21) Appl. No.: 17/628,142

(22) PCT Filed: Jul. 17, 2020
(86) PCT No.: PCT/JP2020/027922

§ 371 Date: Jan. 18, 2022

(30) Foreign Application Priority Data
Jul. 18, 2019 (JP) 2019-132490

(51) Int. Cl. C07K 19/00; C07K 1/107; C12P 19/34

(52) U.S. Cl. CPC C07K 19/00; C07K 1/107; C12P 19/34

(57) ABSTRACT

The present invention provides a peptide that disrupts the self-aggregation of an AtaA polypeptide or separates a bond between the AtaA polypeptide and another subject. A peptide found by the present inventors has the properties of disrupting the self-aggregation of an AtaA polypeptide or separating a bond between the AtaA polypeptide and another subject. A peptide according to the present disclosure includes amino acid sequences AVL, SVL, ATL, or functional equivalent sequences thereof. In one embodiment, a peptide according to the present disclosure has the ability to separate a bond between an AtaA and other molecules (for example, streptavidin and neutral avidin).

### TECHNICAL FIELD

The present disclosure relates to peptides that disintegrate the self-aggregation of AtaA polypeptides, which are adhesive polypeptides, or altered sequences thereof or fragments thereof having self-aggregation properties, and use thereof. The present disclosure further relates to peptides that separate the bond between AtaA polypeptides, or an AtaA polypeptide and another subject, for example, streptavidin or the like, and use thereof.

### BACKGROUND ART

Acinetobacter sp. Tol5 (genus Acinetobacter bacteria Tol5 strain) are non-pathogenic gram-negative bacteria that have a high cell self-aggregation property and exhibits high adhesive properties to various material surfaces, from various hydrophobic plastic carriers to hydrophilic glass and even metal surfaces. The inventors have discovered a novel bacterial nanofiber present on the surface layer of a bacterial cell as a factor which brings about such adhesive properties that have not been reported in other microorganisms, and has further identified a new protein constituting the nanofiber. This protein belongs to the trimeric autotransporter adhesin (TAA) family of Gram-negative bacteria and is named AtaA by the inventors (Non-Patent Literature 1).

AtaA is extremely useful for immobilization of microorganisms and is expected to be applied to various bioprocesses (see, for example, Patent Literatures 1 and 2). Furthermore, in a previous patent application (Patent Literature 3), the inventors reported that various functional peptides and proteins can be introduced into AtaA and presented on the surface of a microorganism. Note that, in Patent Literature 1, AtaA and the gene encoding it (ataA gene) were referred to as AadA and aadA genes, respectively.

### CITATION LIST

### Patent Literature

[PTL 1] WO 2009/104281
[PTL 2] WO 2014/156736
[PTL 3] WO 2015/030171

### Non Patent Literature

[NPL 1] Ishikawa, M.; Nakatani, H.; Hori, K., AtaA, a new member of the trimeric autotransporter adhesins from Acinetobacter sp. Tol 5 mediating high adhesiveness to various abiotic surfaces. PLoS One 2012, 7, (11), e48830.
[NPL 2] Linke, D.; Riess, T.; Autenrieth, I. B.; Lupas, A.; Kempf, V. A., Trimeric autotransporter adhesins: variable structure, common function. Trends Microbiol. 2006, 14, (6), 264-270.

### SUMMARY OF INVENTION

### Solution to Problem

As a result of diligent studies, the inventors have found that a particular tripeptide sequence disintegrates the self-aggregation of the AtaA polypeptide or separates the bond between the AtaA polypeptide fragment and another subject.

Therefore, the present disclosure provides the following.

(1) A peptide comprising an amino acid sequence of AVL, SVL or ATL, or a functional equivalent sequence thereof.
(2) A peptide consisting of an amino acid sequence of AVL, SVL or ATL, or a functional equivalent sequence thereof.
(3) The peptide according to any one of the items above, which has a function of disintegrating the self-aggregation of a polypeptide having the amino acid sequence set forth in SEQ ID NO: 4 or an altered sequence and fragment thereof having a self-aggregation property.
(4) The peptide according to any one of the items above, which has a function of disintegrating the aggregation of cells expressing a polypeptide comprising the amino acid sequence set forth in SEQ ID NO: 4 or an altered sequence thereof or a fragment thereof.
(4B) An attaching and detaching method comprising the steps of: (1) bringing an autotransporter adhesin derived from Acinetobacter microorganisms, such as a microorganism to which a non-specific adhesion property is imparted or enhanced by introducing a DNA encoding the amino acid sequence set forth in SEQ ID NO: 4 or an altered sequence thereof, or a fragment thereof, into contact with a carrier to adhere the microorganism to the carrier; and (2) washing the carrier to which the microorganism is attached in the presence of the peptide according to any one of the items above to desorb the microorganism from the carrier.
(5) The peptide according to any one of the items above, which has a function of separating a bond between a polypeptide comprising the amino acid sequence set forth in SEQ ID NO: 1 or an altered sequence thereof or a fragment thereof, or a fusion or complex of the polypeptide with another amino acid sequence, and another subject.
(6) A composition comprising the peptide according to any one of the items above, for disintegrating self-aggregation of a polypeptide having the amino acid sequence set forth in SEQ ID NO: 4 or an altered sequence and fragment thereof having a self-aggregation property.
(7) A composition comprising the peptide according to any one of the items above or a similar peptide, for separating a bond between a polypeptide comprising the amino acid sequence set forth in SEQ ID NO: 1 or an altered sequence thereof or a fragment thereof, or a fusion or complex of the polypeptide with another amino acid sequence, and another subject.
(8) The composition according to any one of the items above, where the subject is a functionality-impairing entity.
(9) The composition according to any one of the items above, where the subject is streptavidin, neutral avidin or a variant of either thereof.
(10) The composition according to any one of the items above, where the functionality-impairing entity is a cell.
(11) A fusion or complex of the peptide according to any one of the items above and a functionality-impairing entity.
(12) Use of the peptide according to any one of the items above for disintegrating self-aggregation of a polypeptide having the amino acid sequence set forth in SEQ ID NO: 4 or an altered sequence and fragment thereof having a self-aggregation property.
(13) Use of the peptide according to any one of the items above or a similar peptide for separating a bond between a polypeptide comprising the amino acid sequence set forth in SEQ ID NO: 1 or an altered sequence thereof or a fragment thereof, or a fusion or complex of the polypeptide with another amino acid sequence, and another subject.
(14) The use according to any one of the items above, where the subject is a functionality-impairing entity.
(15) The use according to any one of the items above, where the subject is streptavidin, neutral avidin or a variant of either thereof.

The present disclosure is intended so that one or more of the features described above can be provided not only as the explicitly disclosed combinations, but also as other combinations. Additional embodiments and advantages of the present disclosure are recognized by those skilled in the art by reading and understanding the following detailed description as needed.

### Advantageous Effects of Invention

Using the present disclosure, it has become possible to disintegrate the self-aggregation of the AtaA polypeptides and fragments thereof. Therefore, the peptides of the present disclosure can inhibit laminated adhesion due to self-aggregation between AtaA polypeptides, and is thus useful, for example, in aspects where laminated adhesion is not preferable. Furthermore, the peptides of the present disclosure are capable of separating the bond of an AtaA polypeptide fragment to another subject (e.g., a functionality-imparting peptide such as streptavidin or neutral avidin) and is useful in controlling the on/off of processes in bioprocesses in technical fields such as fermentation industry, pharmaceutical industry, chemical product industry, regenerative medicine, cell engineering, medical engineering and biosensors as well as in material engineering processes such as surface modification and material design. Additionally, the peptides of the present disclosure are capable of separating cells (including microorganisms and biofilms) expressing AtaA polypeptides immobilized on the support, from the support, and adjustment of the thickness of the biofilm in the biofilm reactor and the separation from the adherent or fixed state allow cells and carriers to be reused. Furthermore, the peptides of the present disclosure can also be used as the means for isolating the Nhead domain present on the N-terminal side from the AtaA protein expressed on the cell membrane of Acinetobacter.

### BRIEF DESCRIPTION OF DRAWINGS

FIG. 1 shows a still image of the collapse of agglomerates of Acinetobacter sp. Tol5 strain when video shot with a microscope. A shows a microphotograph of the adherent aggregates of Acinetobacter sp. Tol5 strain, with a BS-N medium containing no peptide of the present disclosure delivered thereto. B shows a still image of the Acinetobacter sp. Tol5 strain with 0.1 mM AVL peptide added thereto. C shows a still image with the addition of 0.1 mM ATL peptide. D shows a still image with the addition of 0.1 mM SVL peptide. All photographs are still images photographed within 10 seconds after the medium containing the peptide (for A, the blank medium containing no peptide) was allowed to flow.

FIG. 2 shows separation of the binding between E. coli recombinant Nhead and streptavidin by the peptides of the present disclosure. FIG. 2A shows results of AVL peptide addition. The vertical axis of the graph shows the abundance ratio (volume %) of each particle, while the horizontal axis thereof shows the particle size (nm). Note that the peaks of Nhead and SA after the addition of the peptide overlap with each other.

FIG. 2 shows separation of the binding between E. coli recombinant Nhead and streptavidin by the peptides of the present disclosure. FIG. 2B shows results of ATL peptide addition. The vertical axis of the graph shows the abundance ratio (volume %) of each particle, while the horizontal axis thereof shows the particle size (nm). Note that the peaks of Nhead and SA after the addition of the peptide overlap with each other.

FIG. 2 shows separation of the binding between E. coli recombinant Nhead and streptavidin by the peptides of the present disclosure. FIG. 2C shows results of SVL peptide addition. The vertical axis of the graph shows the abundance ratio (volume %) of each particle, while the horizontal axis thereof shows the particle size (nm). Note that the peaks of Nhead and SA after the addition of the peptide overlap with each other.

FIG. 3 shows the immobilization of biotinylated molecules using the bond between Nhead and streptavidin. The graph shows the detection result by fluorescence of the biotinylated molecules. The vertical axis of the graph shows the fluorescence intensity of 535 nm, while the horizontal axis shows the fluorescence intensity in order from the left, when Nhead is not immobilized, after the first binding reaction, after the first separation reaction, after the second binding reaction, after the second separation reaction, and after the third binding reaction.

### DESCRIPTION OF EMBODIMENTS

The present disclosure is described hereinafter while showing the best mode thereof. Throughout the entire specification, a singular expression should be understood as encompassing the concept thereof in the plural form, unless specifically noted otherwise. Thus, singular articles (e.g., “a”, “an”, “the”, and the like in the case of English) should also be understood as encompassing the concept thereof in the plural form, unless specifically noted otherwise. Further, the terms used herein should be understood as being used in the meaning that is commonly used in the art, unless specifically noted otherwise. Therefore, unless defined otherwise, all terminologies and scientific technical terms that are used herein have the same meaning as the general understanding of those skilled in the art to which the present invention pertains. In case of a contradiction, the present specification (including the definitions) takes precedence.

The definitions of the terms and/or basic technical concepts that are particularly used herein are described hereinafter when appropriate.

### Definitions of Terms

As used herein, “adhere, adhesion” and “immobilize, immobilization” are used interchangeably and used in the sense commonly used in the art, and mean to make at least a portion of the subject of “adhesion” or “immobilization” at least relatively immobile from a certain position on the subject to be adhered or immobilized.

As used herein, “aggregate, aggregation” is used in the sense commonly used in the art and means that polypeptides, cells or the like, in their dispersed state, gather in a mass with the polypeptides themselves, with other polypeptides, with the cells or the like. Particularly when the same type of polypeptide chains aggregate together, it is referred to as “(self-aggregate, self-aggregation)”. For example, as to AtaA, the self-aggregation of multiple AtaA or cells expressing AtaA causes the phenomenon of forming a mass of AtaA peptides or cells; thus, AtaA can be considered as having self-aggregating ability. It has been revealed in the present disclosure that the peptides of the present disclosure can disintegrate the self-aggregation between AtaAs or cells expressing AtaA. For example, the use of the peptides of the present disclosure prevents laminated adhesion from being performed, and thus, it is advantageous in the situation where the laminated adhesion is not preferable.

In addition, applications that maintain the adhesion of AtaA polypeptide but do not allow self-aggregation are useful in the following applications. Specifically, the peptides of the present disclosure can also disperse agglomerates of cells such as microorganisms by disintegrating the self-aggregation between AtaA. Accordingly, the peptides of the present disclosure can also be used in biofilm reactors for applications, such as stripping and cleaning biofilms, and is thus useful in the fermentation industry and biofuel production. Further, the peptides of the present disclosure control the on/off of the bond between the AtaA polypeptide fragment (particularly the fragment that has lost the aggregation property but maintains the adhesive property thereof) and the functionality-impairing entity so that the peptides are useful for controlling the function of the functionality-impairing entity in in bioprocesses in technical fields such as fermentation industry, pharmaceutical industry, chemical product industry, regenerative medicine, cell engineering, medical engineering and biosensors as well as in material engineering processes such as surface modification and material design.

As used herein, “bind, bound” is used in the sense commonly used in the art and means a physical or chemical association between two substances, or a combination thereof. In particular, in the present disclosure, the interaction between AtaA polypeptide and streptavidin, neutral avidin, variants thereof, or the like falls under the “bind” herein. As used herein, “binding” is also referred to as “specific binding” because it corresponds to a specific interaction. On the other hand, “adhesion” or “immobilization” is due to non-specific interactions.

As used herein, “separation, separate” is used in the sense commonly used in the art and means that in two or more substances that are in a “bound” state, at least a part of the “bound” state between the substances is released.

As used herein, “disintegration, disintegrate” is used in the sense commonly used in the art and means that in two or more substances in an “aggregated” state, at least a part of the “aggregated” state between the substances is released.

As used herein, “AtaA” is a protein belonging to the trimeric autotransporter adhesin (TAA) family possessed by Gram-negative bacteria. When “AtaA”, “AtaA peptide”, “a variant of AtaA peptide” and “AtaA polypeptide or a fragment thereof” are referred to herein, unless otherwise specified, they are to encompass not only the full-length AtaA polypeptide, but also variants and fragments of the AtaA polypeptide that have either or both the self-aggregation property and the binding ability to other subjects. AtaA usually has adhesive ability. AtaA forms a homotrimer and has a domain group structure with a signal peptide-Nhead-Nstalk-Chead-Cstalk-Membrane anchor arranged therein in this order from the amino terminal to the carboxy terminal. Respective domain groups are linked by a structure such as a neck or a coiled coil. In addition, Nhead, Nstalk, Chead, and Cstalk include various domains such as Ylhead, Trp-ring, GIN and FGG, and also include some domains repeatedly. Respective domains are linked by a structure such as a neck or a coiled coil. The full-length amino acid sequence and nucleic acid sequence of AtaA are shown in SEQ ID NOs: 4 and 3, respectively. The amino acid sequence and nucleic acid sequence of the fragment sequence of AtaA that does not include a signal peptide at the N-terminal but includes methionine at the beginning are shown in SEQ ID NOs: 1 and 2, respectively. In SEQ ID NOs: 1 and 2, Nhead (including Neck) corresponds to amino acid positions 2 to 258 and nucleic acid positions 4 to 774, Nstalk corresponds to amino acid positions 259 to 2846 and nucleic acid positions 775 to 8538, Chead (not including Neck) corresponds to amino acid positions 2855 to 3093 and nucleic acid positions 8563 to 9279, and Cstalk (including β-barrel inclusions) corresponds to amino acid positions 3094 to 3518 and nucleic acid positions 9280 to 10554. In addition, in the Nhead domain, there are two loop structures that are related to adhesive property and aggregation property and are necessary for binding to the functionality-impairing entity, where these loop structures correspond to amino acid positions 22-50 and 161-175, and nucleic acid positions 64-150 and 481-525, respectively.

As used herein, “Acinetobacter” means the genus Acinetobacter in the taxonomy. The genus Acinetobacter typically includes the Acinetobacter sp. Tol5 strain. The Acinetobacter sp. Tol5 strain is a strain with toluene resolution ability, isolated from the exhaust gas treatment reactor and is available from Katsutoshi Hori Laboratory, Nagoya University. The AtaA peptide or fragments thereof in the present disclosure include, but are not limited to, those produced with the Acinetobacter sp. Tol5 strain belonging to the genus Acinetobacter.

As used herein, the “functionality-impairing entity” is used in the sense commonly used in the art and means those that have any functions or actions. The functionality-impairing entity is bound by the AtaA polypeptide or fragment thereof in the present disclosure. The functions or actions are typically various functions or actions useful in bioprocesses in technical fields such as pharmaceutical industry, chemical product industry, regenerative medicine, cell engineering, medical engineering and biosensors, in materials engineering processes and the like, and include, but are not limited to, functions such as labeling. In one embodiment, the functionality-impairing entity is a protein and/or peptide, or a cell including them, a portion thereof, a biological tissue or organ, or a portion thereof. In a specific embodiment, the functionality-impairing entity is streptavidin, neutral avidin or a variant thereof.

As used herein, “streptavidin” is used in the sense commonly used in the art and means streptavidin produced by the genus Streptomyces, or variants, derivatives or equivalents thereof. Examples of the amino acid sequence thereof include, but are not limited to, P22629-1, and examples of the nucleic acid sequence thereof include, but are not limited to, those specified by X03591. The “streptavidin” herein typically binds to biotin with high affinity. As used herein, “neutral avidin” is used interchangeably with “NeutrAvidin” and the like in the same sense, and means a variant of avidin (particularly, a sugar chain-removed substance) which has a pI value near neutrality and in which non-specific binding is suppressed. “Neutral avidin” also typically binds to biotin with high affinity. “Streptavidin” and “neutral avidin” have the property of specifically binding to the AtaA polypeptide or fragments thereof in the present disclosure.

As used herein, a “corresponding” amino acid or nucleic acid or moiety refer to: an amino acid or nucleotide that has or is expected to have a similar action as a given amino acid or nucleotide or moiety in the polypeptide or polynucleotide to be used as a reference for comparison, in a certain polypeptide or polynucleotide molecule (e.g., AtaA and the like); and for adhesive polypeptides in particular, an amino acid that is present at similar positions in the site required for adhesion or self-aggregation and makes a similar contribution to adhesive property. For example, as for an antisense molecule, it can be a similar part in the ortholog corresponding to a particular part of the antisense molecule. The corresponding amino acid can be, for example, a specific amino acid that is cysteinylated, glutathioneized, S—S bond formed, oxidized (e.g., methionine side chain oxidation), formylated, acetylated, phosphorylated, glycosylated, myristilized, and the like. Alternatively, the corresponding amino acid can be the amino acid responsible for trimerization. Such a “corresponding” amino acid or nucleic acid may be a region or domain over a certain range. Therefore, in such cases, they are referred to herein as a “corresponding” region or domain.

As used herein, “fusion” means that two or more molecules (e.g., polypeptides or nucleic acids) or parts thereof are linked by covalent binding. When two or more moieties are polypeptides and they are covalently bound, they are fusions or fusion products produced by “fusion”, or fusion proteins or fusion polypeptides, which can also be referred to as chimeric polypeptides. Regularly, in the case of a polypeptide fusion, a fusion of nucleic acids is prepared by linking the base sequences of nucleic acids encoding two types of polypeptides, and the fusion is used as a design drawing to produce a fusion polypeptide as a recombinant protein.

As used herein, “complex” is used in the sense commonly used in the art and means any construct including two or more moieties. For example, if one of the moieties is a polypeptide, the other of the moieties may be a polypeptide or any other substances (e.g., sugars, lipids, nucleic acids and other hydrocarbons). Two or more moieties that constitute the complex herein can be bound in a non-covalent manner (e.g., hydrogen bond, ionic bond, hydrophobic interaction, van der Waals force, and the like). Therefore, as used herein, the “complex” refers to molecules formed by binding a plurality of types of molecules, such as polypeptides, polynucleotides, lipids, sugars, and small molecules, by methods other than covalent binding.

In the present disclosure, “functional equivalent sequence” is used for peptides and the like of the present disclosure and means an amino acid sequence or nucleic acid sequence having a function equivalent to the original function. Typical functional equivalent sequences can include, but are not limited to, sequences including conservative substitution amino acids.

In the present disclosure, “conservative substitution” means a substitution in which the hydrophilicity index and/or the hydrophobicity index of the substituting amino acid is similar to that of the original amino acid as described above. Examples of the conservative substitution are well known to those of skill in the art and include, but are not limited to, for example, substitutions within each of the following groups: arginine and lysine; glutamic acid and aspartic acid; serine and threonine; glutamine and asparagine; and valine, leucine, isoleucine, and the like. It is understood that certain sequences of the tripeptides of the present disclosure may typically be conservatively substituted in part or in whole.

The “variant”, “derivative”, “analog” or “mutant” (of proteins, peptides, and the like) as used herein are used interchangeably, and the above terms preferably include molecules including regions that are substantially homologous to the protein or peptide of interest, although not intended to be limiting; and in the case of amino acid sequence, nucleic acid sequence (nucleotide sequence, base sequence), such sequences are referred to as “altered sequence”, “derived sequence”, “analogous sequence”, and “mutated sequence”. Such molecules, in various embodiments, are at least 30%, 40%, 50%, 60%, 70%, 80%, 90%, 95%, or 99% identical, throughout amino acid sequences of the same size, or in comparison with sequences to be aligned by conducting alignment with a computer homology program that is known in the art; or nucleic acids encoding such molecules can hybridize to sequences encoding component proteins under (highly) stringent, moderately stringent, or non-stringent conditions. This means that each is a product of protein alteration by amino acid substitutions, deletions and additions, in which the variant or derivative thereof still exhibits the biological function of the original protein, but not necessarily to the same degree. The variant may have a sequence that is preferably 70% or more identical, more preferably 80% or more identical, even more preferably 90% or more identical, still even more preferably 95% or more identical, 98% or more identical, 99% or more identical, to the original sequence. For example, it is also possible to investigate the biological function of such a protein by appropriate and available in vitro assays described herein or known in the art. “Functionally active” as used herein refers to a polypeptide, i.e., a fragment or derivative, having a structural, regulatory, or biochemical function of protein, such as biological activity, according to aspects to which the polypeptide, i.e., the fragment or derivative, of the present disclosure is associated herein. Accordingly, in one embodiment, the variant of the present disclosure refers to a functional equivalent.

As used herein, “protein”, “polypeptide” and “peptide” are used with the same meaning herein and refer to a polymer of amino acids of any length. As used herein, an amino acid polymer with the amino acids in length of 3, 4, 5, 6, 7, 8, 9 or 10 amino acids is specifically referred to as an “oligopeptide”. The length of the oligopeptide is preferably 3 to 10 amino acids, more preferably 3 to 6 amino acids, and even more preferably 3 to 5 amino acids. The polymer may be linear, branched or cyclic. The amino acids may be natural or non-natural, or may be altered amino acids. The term may also include those assembled into a complex of multiple polypeptide chains. The term also includes naturally or artificially altered amino acid polymers. Such alterations include, for example, disulfide bond formation, glycosylation, lipidation, acetylation, phosphorylation, methylation, trimethylation or any other manipulation or alteration (e.g., conjugation with a labeling component) This definition also includes, for example, polypeptides including one or more analogs of amino acids (including, for example, unnatural amino acids), peptide-like compounds (e.g., peptoids) and other alterations known in the art. As used herein, “amino acid” is a general term for organic compounds having an amino group and a carboxyl group. When the polypeptide according to the embodiments of the present disclosure includes a “specific amino acid sequence”, any amino acid in the amino acid sequence may be chemically modified. In addition, any amino acid in the amino acid sequence may form a salt or a solvate. Furthermore, any amino acid in the amino acid sequence may be L-type or D-type. Even in such cases, the protein according to the embodiments of the present disclosure can be said to include the above “specific amino acid sequence”. As to the chemical modification that an amino acid included in a protein undergoes in vivo, knowns are, for example, N-terminal modification (e.g., acetylation, myristoylation, etc.), C-terminal modification (e.g., amidation, glycosylphosphatidylinositol addition, etc.), side chain modification (e.g., phosphorylation, sugar chain addition, etc.) or the like. The amino acids may be any natural or non-natural amino acids that satisfy the objectives of the present disclosure.

As used herein, peptides having the ability to disintegrate self-aggregation are sometimes referred to as “disintegration peptides”, and peptides that have the ability to separate bonds are referred to as “separated peptides”. While not wishing to be bound by theory, it is understood that even if the peptides have the ability to separate, the peptides may not be able to detach adhesion.

As used herein, “polynucleotide”, “oligonucleotide” and “nucleic acid” are used with the same meaning herein and refer to a polymer of nucleotide of any length. The term also includes an “oligonucleotide derivative” or “polynucleotide derivative”. The “oligonucleotide derivative” or “polynucleotide derivative” refers to an oligonucleotide or polynucleotide that includes a derivative of a nucleotide or has an unusual bond between nucleotides and is used interchangeably. Specific examples of such oligonucleotides include, for example 2′-O-methyl-ribonucleotide, an oligonucleotide derivative in which the phosphate diester bond in the oligonucleotide is converted to a phosphorothioate bond, an oligonucleotide derivative in which the phosphate diester bond in the oligonucleotide is converted to an N3′-P5′phosphodiester bond, an oligonucleotide derivative in which a ribose and a phosphate diester bond in the oligonucleotide are converted into a peptide nucleic acid bond, an oligonucleotide derivative in which uracil in the oligonucleotide is substituted by C-5 propynyl uracil, an oligonucleotide derivative in which uracil in the oligonucleotide is substituted by C-5 thiazole uracil, an oligonucleotide derivative in which cytosine in the oligonucleotide is substituted by C-5 propynylcytosine, an oligonucleotide derivative in which cytosine in the oligonucleotide is substituted by phenoxazine-modified cytosine, an oligonucleotide derivative in which ribose in DNA is substituted by 2′-O-propylribose, and an oligonucleotide derivative in which ribose in the oligonucleotide is substituted by 2′-methoxyethoxyribose. Unless otherwise indicated, the specific nucleic acid sequence is also intended to include sequences that are explicitly shown, as well as the conservatively altered variants (e.g., a degenerate codon substitution) and complementary sequences thereof. Specifically, the degenerate codon substitution can be achieved by creating a sequence in which the third position of one or more selected (or all) codons is substituted by a mixed base and/or deoxyinosine residue (Batzer et al., Nucleic Acid Res. 19:5081(1991); Ohtsuka et al., J. Biol. Chem. 260: 2605-2608(1985); Rossolini et al., Mol. Cell. Probes 8:91-98(1994)). As used herein, “nucleic acid” is also used interchangeably with genes, cDNAs, mRNAs, oligonucleotides, and polynucleotides. As used herein, “nucleotide” may be natural or non-natural.

As used herein, “gene” means a factor that defines a genetic trait, and “gene” may also refer to “polynucleotide”, “oligonucleotide” and “nucleic acid”.

As used herein, “homology” of a gene means the degree of identity of two or more gene sequences to each other, and generally, having “homology” means a high degree of identity or similarity. Thus, as the homology of two certain genes increases, the identity or similarity of the sequences thereof increases. Whether or not two types of genes are homologous can be determined by direct comparison of the sequences, or in the case of nucleic acids, can be examined by hybridization methods under stringent conditions. In direct comparison of two gene sequences, the genes thereof are homologous if the DNA sequences are typically at least 50% identical, preferably at least 70% identical, more preferably at least 80%, 90%, 95%, 96%, 97%, 98% or 99% identical, between the gene sequences. Similarly, the concept of “homology” also applies to amino acid sequences, in which case the numerical values of identity etc. can be interpreted by replacing them with amino acid sequences. Accordingly, as used herein, “homolog” or “homologous gene product” means a protein in another species, preferably a microorganism, more preferably a bacterium, which exerts the same biological function as the protein component of the complex to be further described herein. Such a homolog is also sometimes referred to as an “ortholog gene product”. It is understood that such a homolog, homologous gene product, ortholog gene product, etc. can also be used as long as they meet the objectives of the present disclosure.

Amino acids may be mentioned herein by either their commonly known three letter symbols or their one character symbols recommended by the IUPAC-IUB Biochemical Nomenclature Commission. Similarly, nucleotides may be mentioned by their commonly recognized one character codes. Comparison of similarity, identity, and homology of an amino acid sequence and a base sequence is calculated herein by using a sequence analysis tool BLAST with default parameters. For example, identity can be searched using BLAST 2.7.1 (published on Oct. 19, 2017) of the NCBI. Herein, values for “identity” generally refer to a value obtained when aligned under the default conditions using BLAST. However, when a higher value is obtained by changing a parameter, the highest value is considered the value of identity. When identity is evaluated in a plurality of regions, the highest value thereamong is considered the value of identity. “Similarity” is a value calculated by taking into consideration a similar amino acid in addition to identity.

In one embodiment of the present disclosure, the “several” may be, for example, 10, 8, 6, 5, 4, 3, or 2, or less than or equal to any of these values. It is known that a polypeptide with one or several amino acid residue deletions, additions, insertions or substitutions with other amino acids, still maintains its biological activity (Mark et al., Proc Natl Acad Sci USA. 1984 September; 81(18): 5662-5666., Zoller et al., Nucleic Acids Res. 1982 Oct. 25; 10(20): 6487-6500., Wang et al., Science. 1984 Jun. 29; 224(4656): 1431-1433.). Peptides with deletions and the like can be prepared, for example, by a site-specific mutagenesis method, a random mutagenesis method, or the like. As a site-specific mutagenesis method, for example, KOD-Plus-Mutagenesis Kit (TOYOBO CO., LTD.) can be used. It is possible to select a polypeptide having the same activity as the wild-type from the mutant-type polypeptides in which a deletion or the like has been introduced, by performing various characterizations such as FACS analysis and ELISA.

In one embodiment of the present disclosure, the numerical value of identity or the like, i.e., “70% or greater”, can be, for example, 70% or greater, 75% or greater, 80% or greater, 85% or greater, 90% or greater, 95% or greater, 96% or greater, 97% or greater, 98% or greater, 99% or greater, or 100% or greater, or within a range between any two of numerical values of such starting points. The above “identity” is computed by computing the ratio of the number of amino acids homologous in two or more amino acid sequences in accordance with a known method described above. Specifically, amino acid sequences in a group of amino acid sequences to be compared are aligned before computing the ratio, and a space is introduced in a part of the amino acid sequences if needed to maximize the ratio of identical amino acids. A method for alignment, ratio computation method, comparison method, and computer program associated therewith are conventional and well known in the art (e.g., aforementioned BLAST or the like). As used herein, “identity” and “similarity” can be represented by a value measured by NCBI's BLAST, unless specifically noted otherwise. Blastp can be used with the default settings as the algorithm for comparing amino acid or base sequences with BLAST. Measurement results are quantified as Positives or Identities.

As used herein, a “polynucleotide that hybridizes under stringent conditions” refers to well-known conditions commonly used in the art. Such a polynucleotide can be obtained by using a polynucleotide selected from the polynucleotides of the present disclosure as a probe and using a colony hybridization method, a plaque hybridization method or a southern blot hybridization method. Specifically, the above means such a polynucleotide that can be identified by performing hybridization at 65° C. in the presence of 0.7 to 1.0 M NaCl using a filter with immobilized DNA from a colony or plaque, followed by washing the filter under 65° C. conditions with a 0.1 to 2-fold concentration SSC (saline-sodium citrate) solution (where the composition of the 1-fold concentration SSC solution is 150 mM sodium chloride and 15 mM sodium citrate). For the “stringent conditions”, the conditions below, for example, can be adopted: (1) use of low ionic strength and high temperature for washing (e.g., at 50° C., 0.015M sodium chloride/0.0015M sodium citrate/0.1% sodium dodecyl sulfate); (2) use of a denaturing agent such as formamide during hybridization (e.g., at 42° C., 50% (v/v) formamide and 0.1% bovine serum albumin/0.1% ficoll/0.1% polyvinylpyrrolidone/50 mM and pH 6.5 sodium phosphate buffer, and 750 mM sodium chloride, 75 mM citrate sodium), or (3) incubation overnight at 37° C. in a solution containing 20% formamide, 5×SSC, 50 mM sodium phosphate (pH 7.6), 5×denhard solution, 10% dextran sulfate, and 20 mg/ml denatured sheared salmon sperm DNA, followed by washing the filter with 1×SSC at about 37 to 50° C. Note that the formamide concentration may be 50% or higher. Washing time may be 5, 15, 30, 60, or 120 minutes, or longer. Multiple factors such as temperature and salt concentration can be considered as factors that affect the stringency of the hybridization reaction, and for details, Ausubel et al., Current Protocols in Molecular Biology, Wiley Interscience Publishers, (1995) may be referenced. Examples of “highly stringent conditions” are 0.0015M sodium chloride, 0.0015M sodium citrate, 65 to 68° C., or 0.015M sodium chloride, 0.0015M sodium citrate, and 50% sodium citrate, at 42° C. Hybridization can be performed in accordance with the method described in experimental publications such as Molecular Cloning 2nd ed., Current Protocols in Molecular Biology, Supplement 1-38, DNA Cloning 1: Core Techniques, A Practical Approach, Second Edition, Oxford University Press (1995). In this regard, sequences containing only the A sequence or only the T sequence are preferably excluded from the sequences to be hybridized under stringent conditions. Moderate stringent conditions can be easily determined by one of ordinary skill in the art on the basis of, for example, the length of the DNA, shown in Sambrook et al., Molecular Cloning: A Laboratory Manual, No. 3, Vol. 1, 7. 42-7. 45 Cold Spring Harbor Laboratory Press, 2001; and as to nitrocellulose filters, included are the use of: hybridization conditions with a pre-cleaning solution of 5×SSC, 0.5% SDS, and 1.0 mM EDTA (pH 8.0), and at about 40 to 50° C., about 50% formamide, 2×SSC-6×SSC (or other similar hybridization solution, such as Stark's solution, in about 50% formamide at about 42° C.); and washing conditions of about 60° C., 0.5×SSC, 0.1% SDS. Accordingly, the peptides used in this disclosure also include polypeptides coded by nucleic acid molecules that hybridize under highly or moderately stringent conditions to nucleic acid molecules that code the polypeptides specifically described in the present disclosure.

The peptides of the present disclosure may preferably be “purified” or “isolated”. A “purified” substance or biological factor (e.g., nucleic acid or protein) as used herein refers to one in which at least some of the factors naturally associated with the substance or biological factor have been removed. Thus, the purity of the biological factor in the purified biological factor is usually higher (i.e., enriched) than the purity of the biological factor in the state in which the biological factor is normally present. The term, “purified”, as used herein means that there is preferably at least 75% by weight, more preferably at least 85% by weight, even more preferably at least 95% by weight, and most preferably at least 98% by weight, of the same type of biological factor present. The substance or biological factor used in the present disclosure is preferably a “purified” substance. An “isolated” substance or biological factor (e.g., nucleic acid or protein) as used herein refers to one in which the factors naturally associated with the substance or biological factor have been substantially removed. The term, “isolated”, as used herein does not necessarily have to be expressed in purity as it varies in accordance with the objective thereof, but if necessary, means that there is preferably at least 75% by weight, more preferably at least 85% by weight, even more preferably at least 95% by weight, and most preferably at least 98% by weight, of the same type of biological factor present. The substance used in the present disclosure is preferably an “isolated” substance or biological factor.

As used herein, “fragment” refers to a polypeptide or polynucleotide with a sequence length of 1 to n−1 with respect to a full-length polypeptide or polynucleotide (with the length of n). The length of the fragment can be varied as appropriate according to the objective thereof, and the lower limit of the length thereof, in the case of polypeptides, includes, for example, 3, 4, 5, 6, 7, 8, 9, 10, 15, 20, 25, 30, 40, 50 and more amino acids; and lengths represented by integers not specifically listed here (e.g., 11) can also be appropriate as lower limits. Further, in the case of polynucleotides, included are 5, 6, 7, 8, 9, 10, 15, 20, 25, 30, 40, 50, 75, 100 and more nucleotides; and lengths represented by integers not specifically listed here (e.g., 11) can also be appropriate as lower limits. As used herein, it is understood that such a fragment falls within the scope of the present disclosure, for example, if the full length one functions as a separable active molecule, as long as the fragment itself also functions as a peptide.

As used herein, “biological function” refers to a specific function that a certain gene or a nucleic acid molecule or polypeptide associated therewith may have in vivo or in vitro in the context of the gene, nucleic acid molecule or polypeptide, the examples of which include, but are not limited to, for example, a disintegration function, a separation function and the like. As used herein, the biological function can be exerted by the corresponding “biological activity”. As used herein, “biological activity” refers to the activity that a factor (e.g., polynucleotide, protein, etc.) may have, and includes activities that exert various functions (e.g., a disintegration function and a separation function). The “biological activity” may be an activity exerted in vivo or an activity exerted in vitro by secretion or the like. For example, if a certain factor is an enzyme, the biological activity thereof includes the enzyme activity thereof. Such biological activity can be measured by techniques well known in the art. Thus, “activity” refers to various measurable indicators that indicate or reveal binding (either direct or indirect) or various measurable indicators that affect the response (i.e., have a measurable effect in response to some exposure or stimulus), which may also include, for example, affinities for compounds that directly bind to the polypeptides or polynucleotides of the present disclosure, or for example, the amount of upstream or downstream protein after some stimulus or event, or other similar scales of function.

As used herein, “expression” of a gene, polynucleotide, polypeptide or the like refers to the gene or the like to undergo a certain action in vivo thus turning into a different form. This preferably refers to a gene, a polynucleotide or the like to be transcribed and translated into the form of a polypeptide; however, it is also an aspect of the expression that they are transcribed to produce mRNA. Accordingly, the “expression product” as used herein includes such a polypeptide or protein, or mRNA. More preferably, the form of such a polypeptide may be the one that is post-translationally processed. For example, the expression level of a peptide can be determined by any method. Specifically, assessing the amount of mRNA encoding the peptide, the amount of the peptide, and the biological activity of the peptide allows finding out of the expression level of the peptide. The amount of mRNA encoding the peptide, polypeptide or protein can be determined by methods as detailed elsewhere herein or by other methods known in the art.

As used herein, “similarity”, “functional equivalence”, or “functional equivalent” means any entity that has the same objective function but a different structure with respect to the original entity of interest. Thus, it is understood that the functional equivalent of the peptide of the present disclosure is not the peptide of the present disclosure itself, but includes a mutant or variant thereof (e.g., an amino acid sequence variant or the like), which has a biochemical action of the peptide, such as the function to separate bonds or the function to disintegrate self-aggregation as well as such a functional equivalent (including, for example, a nucleic acid encoding the mutant or variant, a vector or a cell including the nucleic acid, and the like) which, at the time of action, can be transformed into a mutant or variant that has the biochemical action of the peptide. In the present disclosure, it is understood that the functional equivalent of the peptide can be used in the same manner as the original peptide, without special mention. Functional equivalents can be found by searching databases and the like. As used herein, “searching” refers to finding another nucleic acid base sequence with a particular function and/or property by utilizing a certain nucleic acid base sequence electronically, biologically or otherwise. Examples of electronic searching include, but are not limited to, BLAST (Altschul et al., J. Mol. Biol. 215: 403-410(1990)), FASTA (Pearson & Lipman, Proc. Natl. Acad. Sci., USA 85:2444-2448 (1988)), Smith and Waterman method (Smith and Waterman, J. Mol. Biol. 147:195-197 (1981)), and Needleman and Wunsch method (Needleman and Wunsch, J. Mol. Biol. 48:443-453 (1970)). Examples of biological searching include, but are not limited to, stringent hybridization, microarrays with genomic DNA attached to nylon membranes, etc. or microarrays attached to glass plates (microarray assays), PCR and in situ hybridization. Herein, it is intended that the genes used in the present disclosure should also include the corresponding genes identified by such electronic and biological searching.

As for the functional equivalent of the present disclosure (polypeptide, etc.), it is possible to use amino acid sequences in which one or more amino acids are inserted, substituted or deleted, or added to one or both ends. As used herein, “one or more amino acids are inserted, substituted or deleted, or added to one or both ends” means that alterations have been made with the substitution of a plurality of amino acids that may occur naturally, by well-known technical methods such as site-specific mutagenesis methods, or by natural mutations. An altered amino acid sequence can have, for example, 1 to 30, preferably 1 to 20, more preferably 1 to 9, still more preferably 1 to 5, and particularly preferably 1 to 2 amino acids added to one or both ends thereof. The altered amino acid sequence may be preferably such an amino acid sequence that has one or more (preferably one or several, or 1, 2 or 3) conservative substitutions in the amino acid sequence of AVL, ATL or SVL. “Conservative substitution” herein means a substitution of one or more amino acid residues with other chemically similar amino acid residues so as not to substantially alter the function of the peptide. Examples thereof include substitutions of a hydrophobic residue with another hydrophobic residue, substitutions of a polar residue with another polar residue having the same charge, and the like. Functionally similar amino acids that can be substituted in this manner are known in the art for each amino acid. Specific examples include alanine, valine, isoleucine, leucine, proline, tryptophan, phenylalanine, methionine, and the like for nonpolar (hydrophobic) amino acids, and glycine, serine, threonine, tyrosine, glutamine, asparagine, cysteine, and the like for polar (neutral) amino acids. Examples of positively charged (basic) amino acids include arginine, histidine, lysine, and the like. Further, examples of a negatively-charged (acidic) amino acid include aspartic acid, glutamic acid, and the like.

As used herein, “support” refers to a substance capable of carrying a polypeptide, protein, polynucleotide, cell, bacterium, or virus. The material for use as a support can be any material that can form a solid surface, and examples thereof include, but are not limited to, a lipid double structure (e.g., liposome), glass, silica, silicon, ceramic, silicon dioxide, plastic, metal (including alloy), titanium, acrylic, natural and synthetic polymer (e.g., polystyrene, polypropylene, polyethylene, polyvinyl chloride, polyurethane, cellulose, chitosan, dextran, nylon, polycarbonate, polylactic acid, and polytetrafluoroethylene (PTFE)).

As used herein, “kit” refers to a unit providing parts to be provided (e.g., an enzyme, buffer, user manual, and the like) which are generally separated into two or more compartments. Such a kit form is preferred when providing a composition, which should not be provided in a mixed state for stability or the like and is preferably used by mixing immediately prior to use. Such a kit advantageously comprises an instruction or user manual describing how the provided parts (e.g., peptides) are used or how a reagent or waste fluid after use should be processed. When a kit is used as a reagent kit herein, the kit generally comprises an instruction or the like describing the method of use of the enzyme, or the like.

As used herein, “instruction” is a document with an explanation of the method of use of the present disclosure for users. The instruction provides description for instructing the method of use of the present disclosure. If required, the instruction is prepared in accordance with a format specified by a regulatory authority of the country in which the present invention is practiced (e.g., Ministry of Health, Labour and Welfare, Ministry of Agriculture, Forestry and Fisheries, or the like in Japan, Food and Drug Administration (FDA) or Department of Agriculture (USDA) in the U.S., or the like), with an explicit description showing approval by the regulatory authority. An instruction can be provided in, but not limited to, paper media. An instruction can also be provided in a form such as electronic media (e.g., websites provided on the Internet or emails).

### Preferred Embodiments

Preferred embodiments of the present disclosure are described below. Embodiments provided below are provided to facilitate the understanding of the present disclosure. It is understood that the scope of the present disclosure should not be limited to the following descriptions. Thus, it is apparent that those skilled in the art can make appropriate modifications within the scope of the present disclosure by referring to the descriptions herein. It is understood that the following embodiments of the present disclosure can be used alone or in combination.

(Peptide with Disintegration or Separation Function)

In one aspect, the present disclosure provides peptides comprising the amino acid sequences of AVL, SVL or ATL or altered sequences thereof (particularly functional equivalent sequences). Herein, it is understood that the “functional equivalent sequence” includes a peptide having any sequence with the ability to disintegrate the self-aggregation of AtaA, or the ability to separate the binding of an AtaA fragment to another subject (e.g., functionality-impairing molecules, such as streptavidin and neutral avidin), of AVL, SVL or ATL peptides. Preferably, the peptides of the present disclosure include an amino acid sequence of AVL, SVL or ATL. More preferably, the peptides of the present disclosure consist of an amino acid sequence of AVL, SVL or ATL.

The peptides of the present disclosure may be in the form of oligopeptides including an amino acid sequence of AVL, SVL or ATL or altered sequences thereof. The above oligopeptides may be such oligopeptides in which any 1, 2, 3, 4, 5, 6, 7 or more amino acids are added to the N-terminus, C-terminus, or both of the amino acid sequences of AVL, SVL or ATL or altered sequences thereof. Preferably, amino acids added to the N-terminus, C-terminus, or both are amino acids with short side chains (e.g., Gly, Ala, Val, Leu and Ile, etc.) or amino acids with no side chain charge (amino acids other than Glu, Asp, Lys, His and Arg).

In a preferred embodiment, this peptide consists of an amino acid sequence of AVL, SVL or ATL or a functional equivalent sequence thereof. In such a preferred embodiment, a short peptide referred to as a tripeptide surprisingly exerts the ability to separate the binding of AtaA to other molecules (e.g., streptavidin and neutral avidin).

In one aspect, the present disclosure provides a composition for separating the binding of AtaA or a variant thereof to another molecule. This composition contains a peptide comprising the amino acid sequence of AVL, SVL or ATL or a functional equivalent sequence thereof. Preferably, the peptides of the present disclosure include the amino acid sequence of AVL, SVL or ATL. More preferably, the peptides contained in this composition are an AVL, SVL or ATL tripeptide.

In one preferred embodiment, the AtaA or a variant thereof or fragment thereof, which is the subject of disintegration of self-aggregation or separation of binding by the peptides of the present disclosure, may be any embodiment of the “AtaA polypeptide” described herein. In one embodiment, the peptides of the present disclosure are used to disintegrate an agglomerate of AtaA or a variant thereof. Disintegration of the agglomerate facilitates the isolation, purification, etc., of AtaA or a variant thereof. In addition, the peptides of the present disclosure are used to disintegrate the aggregation of cells expressing a polypeptide including the amino acid sequence set forth in SEQ ID NO: 4 or an altered sequence thereof or a fragment thereof. Thus, the peptides of the present disclosure can disintegrate microbial agglomerates and biofilms formed by AtaA or a variant thereof. In another embodiment, the peptides of the present disclosure are used to separate the binding of the AtaA fragment or a variant thereof set forth in SEQ ID NO: 1 to another subject.

In one embodiment, the present disclosure provides a method for attaching and detaching microorganisms, the method including the steps of: (1) bringing an autotransporter adhesin derived from Acinetobacter microorganisms, such as a microorganism to which a non-specific adhesion property is imparted or enhanced by introducing a DNA encoding the amino acid sequence set forth in SEQ ID NO: 4 or an altered sequence thereof, or a fragment thereof, into contact with a carrier to adhere the microorganism to the carrier; and (2) washing the carrier to which the microorganism is attached in the presence of the peptide of the present disclosure (e.g., the peptide according to the item (1) or (2) of the present disclosure) to desorb the microorganism from the carrier. As for the attaching and detaching method, for example, approaches described in Japanese Laid-Open Publication No. 2017-55676 and the like can be utilized. As used herein, “attaching and detaching (method)” refers to a method including the step (operation) of attaching a microorganism to a carrier and the step (operation) of desorbing the microorganism attached to the carrier.

In the above step (1), i.e., the attaching step, prepared first are microorganisms (also referred to herein as “adhesive property-imparting microorganisms”) in which a non-specific adhesive property has been imparted or enhanced by the introduction of DNA (e.g., a sequence encoding the amino acid sequence set forth in SEQ ID NO: 4 or an altered sequence thereof, or a fragment thereof; also referred to as “adhesive property-imparting DNA” herein) encoding AtaA derived from Acinetobacter microorganisms. Examples of the adhesive property-imparting DNA are, preferably, sequences encoding the amino acid sequence set forth in SEQ ID NO: 4 or an altered sequence thereof, or a fragment thereof, which is any AtaA polypeptide described in detail in other parts of the present specification, isolated or identified from Acinetobacter sp. Tol5 strains.

Various microorganisms can be used as the microorganisms into which adhesive property-imparting DNA is introduced (target microorganisms). The target microorganisms are not particularly limited, and examples thereof include microorganisms having no or weak non-specific adhesive property. The target microorganisms may be any of a wild strain, a mutant strain, and a recombinant strain. Appropriate microorganism are selected in accordance with objectives of the present invention. Examples of microorganisms that can be used as the target microorganisms include: bacteria of the genus Escherichia, such as Escherichia coli; bacteria of the genus Acinetobacter, such as Acinetobacter calcoaceticus; bacteria of the genus Ralstonia, such as Ralstonia eutropha; bacteria of the genus Pseudomonas, such as Pseudomonas putida or Pseudomonas fluorescens; bacteria of the genus Aeromonas, such as Aeromonas caviae; bacteria of the genus Alcaligenes, such as Alcaligenes latus; bacteria of the genus Xanthomonas, such as Xanthomonas campestris; bacteria of the genus Desulfomonile, such as Desulfomonile tiedjei; bacteria of the genus Desulfuromonas, such as Desulfuromonas chloroethenica; bacteria of the genus Chromobacterium, such as Chromobacterium chocolatum; bacteria of the genus Burkholderia, such as Burkholderia arboris; bacteria of the genus Rhodobacter; bacteria of the genus Acidovorax, such as Acidovorax facilis; and bacteria of the genus Zymomonas, such as Zymomonas mobilis. Introduction of the adhesive property-imparting DNA into the target microorganisms and transformation thereof allows obtaining of microorganisms with the non-specific adhesive property imparted thereto or enhanced therein. Typically, ligating the adhesive property-imparting DNA to a suitable vector and transforming the target microorganisms (host microorganisms) with the vector allows obtaining of microorganisms with the non-specific adhesive property imparted thereto or enhanced therein. Specifically, the vector was prepared by ligating the DNA under the control of a constitutively expressed promoter or by ligating the DNA under the control of an inducible expression system promoter. Transforming the host microorganisms with this vector allows obtaining of microorganisms with the non-specific adhesive property imparted thereto or enhanced therein.

In the step (1), the prepared adhesive property-imparting microorganisms are brought into contact with a carrier. The contact here is carried out under conditions where the adhesive property-imparting microorganisms can adhere to the carrier. The adhesive property-imparting microorganisms have high adhesion and exhibit adhesive properties to various carriers under normal culture conditions. Therefore, except in special cases (typically, when the desorption treatment liquid adopted in the step (2) described below is used as a medium, or under low ionic strength (see International Publication No. WO 2014/156736 Pamphlet)), the adhesive property-imparting microorganisms adhere to the carrier by contact.

The adhesive property-imparting microorganisms adhered to the carrier in the step (1) are usually subjected to one or more treatments or reactions (for details of the treatments or reactions, see Japanese Laid-Open Publication No. 2017-55676 for example). In the present disclosure, the step (2), that is, the desorption step is subsequently performed. In the step (2), the adhesive property-imparting microorganisms adhered to the carrier are treated with a solution containing the separated peptide according to the present disclosure that meets predetermined conditions so that the adhesive property-imparting microorganisms can be desorbed from the carrier.

In one embodiment, other molecules are streptavidin, neutral avidin or variants thereof. They are useful because the separation and binding can be freely manipulated in various test systems using streptavidin, neutral avidin or variants thereof.

In one typical embodiment, the present disclosure provides a composition for disintegrating self-aggregation of polypeptides having the amino acid sequence set forth in SEQ ID NO: 4 or an altered sequence and fragment thereof having a self-aggregation property.

In one embodiment, the other molecules mentioned above are functionality-impairing entities. Examples of the functionality-impairing entities include, but are not limited to, such entities having functions useful in biosensors, separation and labeling of biomolecules, regenerative medicine, production of bio, organic or inorganic hybrid materials, surface modification and functionalization, fermentation industry, pharmaceutical industry, chemical product industry, and the like. In a specific embodiment, functionality-impairing entities are proteins and/or peptides. In a more specific embodiment, functionality-impairing entities are streptavidin, neutral avidin, derivatives thereof, enzymes, antibodies, fluorescent molecules, receptors, functional peptides (antibacterial, fluorescent and signaling factors, inducing factors, etc.), nucleic acid aptamers, serum components, or cells. In a specific embodiment, the peptides of the present disclosure are provided as a complex or fusion with the above functional molecules.

(AtaA Polypeptides)

The peptides of the present disclosure are useful for disintegrating the self-aggregation of a full-length AtaA polypeptide consisting of the amino acid sequence set forth in SEQ ID NO: 4, or a polypeptide having an altered sequence and fragment thereof having a self-aggregation property.

The peptides of the present disclosure are also useful for disintegrating the aggregation of cells expressing a full-length AtaA polypeptide consisting of the amino acid sequence set forth in SEQ ID NO: 4 or a polypeptide including an altered sequence or fragment thereof having a self-aggregation property. The cells expressing the AtaA polypeptide or an altered sequence or fragment thereof may be bacterial cells expressing AtaA polypeptides by means, such as vectors, or other bacterial cells as well as the cells that naturally express AtaA such as the Acinetobacter sp. Tol5 strain.

(Production of Proteins or Peptides)

The proteins and peptides of the present disclosure can be produced by expression systems using microorganisms, or chemical synthesis.

Needless to say, however, in the case of short peptides such as tripeptides, these peptides can also be synthesized by peptide synthesis methods well known in the art.

(Use of Peptides)

In one aspect, the peptides of the present disclosure are useful in materials engineering processes. In another aspect, the disintegration peptides and separated peptides of the present disclosure are useful in industries that use bioprocesses. In a certain embodiment, the disintegration peptides and separated peptides of the present disclosure disintegrate the self-aggregation of the AtaA polypeptides to densely consolidate and immobilize the proteins, peptides or microorganisms, thus enabling material engineering processes such as surface modification and material design as well as processes in bioprocesses in technical fields such as fermentation industry, pharmaceutical industry, chemical product industry, regenerative medicine, cell engineering, medical engineering and biosensors, to be carried out efficiently. Additionally, the peptides of the present disclosure are capable of separating cells (including microorganisms and biofilms) expressing AtaA polypeptides immobilized on the support, from the support, where adjustment of the thickness of the biofilm in the biofilm reactor and the separation from the adherent or fixed state allow cells and carriers to be reused.

(Production of AtaA Polypeptides)

In one embodiment, the adhesive polypeptides and fusion polypeptides of the present disclosure are produced by expression systems using microorganisms. The adhesive polypeptides or fusion polypeptides thereof of the present disclosure, can be produced in E. coli as recombinant proteins in the manner commonly used in laboratories. That is, a gene encoding a fragment of AtaA or a fusion peptide obtained by fusing a peptide to the fragment is inserted at an appropriate position of an appropriate E. coli expression vector, such as the pET system, to transform E. coli, followed by adding an inducer to induce gene expression or to constitutively express by constitutive expression promotion. The protein is often produced within the cytoplasm; however, the protein can also be secreted and produced in the periplasm. In the latter case, the gene sequence encoding the signal peptide originally possessed by the AtaA protein or the signal peptide integrated into the E. coli expression vector is coupled to the 5′end side encoding the protein so that the protein can be produced in the form of a signal peptide added to the amino-terminal side of the protein. Note that the signal peptide is cleaved and removed when the produced protein is secreted into the periplasm; thus, the resulting AtaA fragment or fusion protein thereof does not contain a signal peptide. The protein produced within cells is usually separated and purified from the solubilizing component, but may be obtained as an active protein by unwinding after its separation from the inclusion body. Note that the produced protein can be separated and purified by a commonly used affinity purification method using a His tag or Strep tag fused to the protein in advance. Furthermore, when a SNAP tag or the like is fused thereto, a molecule having a benzylguanyl group may be used for separation and purification. To further increase the degree of purification, ion exchange chromatography or gel filtration may be used in combination. In the production method of adhesive polypeptides using this microorganism, detailed conditions, such as the method for expressing adhesive polypeptides, the host for expressing adhesive polypeptides and the method for purifying adhesive polypeptides secreted from the host, are to be appropriately adjusted by those skilled in the art. Furthermore, the adhesive polypeptides of the present disclosure can also be expressed using various expression systems such as, secretory production systems and cell-free expression systems, in addition to the intrabacterial expression systems of microorganisms.

The method for causing the above-mentioned microorganisms to produce the polypeptides of interest including the adhesive polypeptides of the present disclosure is merely exemplary, and those skilled in the art can prepare synthetic genes using a method commonly used in the art, while referring to the nucleic acid sequences (base sequences) or amino acid sequences of the genes of the present disclosure, thereby readily performing the design and construct of a DNA fragment to be inserted into the expression vector.

(General Technology)

The molecular biological methodology, biochemical methodology, and microbiological methodology used herein are well known and commonly used in the art, which are described, for example, in Sambrook J. et al. (1989). Molecular Cloning: A Laboratory Manual, Cold Spring Harbor and its 4th Ed. (2012), Masato Okada, Kaori Miyazaki, 2011, “Protein Experiment Notes, Revised 4th Edition”, First and Second Volumes, Yodosha Co., Ltd., or the like. Relevant portions thereof (which may be the entire document) are incorporated herein by reference.

(Note)

As used herein, “or” is used when “at least one or more” of the listed matters in the sentence can be employed. When explicitly described herein as “within the range” of “two values”, the range also includes the two values themselves.

Reference literatures such as scientific literatures, patents, and patent applications cited herein are incorporated herein by reference to the same extent that the entirety of each document is specifically described.

As described above, the present disclosure has been described while showing preferred embodiments to facilitate understanding. The present disclosure is described hereinafter based on Examples. The above descriptions and the following Examples are not provided to limit the present disclosure, but for the sole purpose of exemplification. Thus, the scope of the present disclosure is not limited to the embodiments and Examples specifically described herein and is limited only by the scope of claims.

### EXAMPLES

The Examples are described hereinafter. When required, organisms used in the following Examples were handled in compliance with the guidelines stipulated by the Nagoya University, regulatory agency, or the Cartagena Protocol. For reagents, the specific products described in the Examples were used. However, the reagents can be substituted with an equivalent product from another manufacturer (Sigma-Aldrich, Fuji Film, Wako Pure Chemical, Nacalai Tesque, R & D Systems, USCN Life Science INC, Thermo Fisher Scientific, Kanto Chemical, Funakoshi, Tokyo Chemical Industry, Merck, or the like).

The Nhead recombinant protein used in the following examples was prepared as follows.

### Manufacture Examples

The construct design of the Nhead recombinant protein is shown in SEQ ID NO: 5. The Nhead recombinant protein designed in this way expresses a recombinant polypeptide corresponding to the amino acid fragment at positions 59-325 of the AtaA sequence: the amino acid fragment at positions 2-268 of SEQ ID NO: 1. To prepare this construct, a gene fragment encoding amino acid 1-268 positions of SEQ ID NO: 1 was inserted into the restriction enzyme XbaI/BsaI digestion fragment of the pIBA-GCN4tri-His vector, thus preparing pIBA::Nhead recombinant DNA (pIBA-GCN4tri-His::ataA₅₉₋₃₂₅).

Cultures obtained by culturing BL21 star (DE3; pIBA-GCN4tri-His::ataA₅₉₋₃₂₅) overnight were diluted 1: 100 in a LB medium and incubated at 37° C. for 3 hours. After the incubation, 0.2 μg/ml anhydrous tetracycline (AHTC) was added to the medium, followed by further incubation at 28° C. for 6 hours. The cells were harvested by centrifugation at 5,000×g for 15 minutes at 4° C. and resuspended in a lysis buffer (20 mM Tris-HCl, 150 mM NaCl and 20 mM imidazole, pH 9.0), followed by dissolving at 1,000 bar for 10 minutes using a high-pressure homogenizer (LAB 2000, SMT Co., Ltd.). After centrifugation at 10,000×g for 15 minutes at 4° C., the supernatant was loaded onto a Ni-NTA Superflow column (Qiagen NV) and washed twice with lysis buffer to remove unbound protein. The bound protein was eluted with an elution buffer (20 mM Tris-HCl, 150 mM NaCl and 400 mM imidazole, pH 9.0). The eluted protein was loaded onto an anion exchange column (HiTrap Q HP, GE Healthcare) and the flow-through fraction containing the recombinant protein was dialyzed against 50 mM phosphate buffer (pH 6.0). The recombinant protein was then purified using a cation exchange column (HiTrap SP HP, GE Healthcare) in a phosphate buffer (pH 6.0) with a linear concentration gradient of 0 to 1,000 mM NaCl. The peak fraction containing the recombinant protein was concentrated by ultrafiltration using a centrifugal filter device (Amicon ultra, EMD Millipore), loaded onto a gel filtration column (HiLoad 26/60 Superdex 200 pg, GE Healthcare) equilibrated with 25 mM Tris-HCl (pH 9.0), and purified by gel filtration. Those prepared by the above procedure were used in the following examples.

### Example 1: Characteristics of Agglutinates of Acinetobacter sp. Tol5 Strain

In this example, microscopic observation of agglutinates of Acinetobacter sp. Tol 5 strain was performed.

(Method)

(Preparation of Flow Cell)

The basic structure of the flow cell system constructed in the present disclosure is a silicone tube, for delivering a cell suspension or fluid, to which a glass tube is connected for observing cells. To construct a rectangular parallelepiped flow cell system, a silicone tube with a length of 300 mm, an inner diameter of 1 mm, and an outer diameter of 2 mm was connected to either end of rectangular parallelepiped glass with a length of 50 mm and each inner diameter of 1 mm (Vitrocom), where the connections were sealed with paraffin film. The silicone tube at the entrance was connected to a syringe pump (Legato 200, KD Scientific) via a three-way stopcock (Terumo Corporation), and the liquid was sent to the cell observation part (flow cell) of the rectangular parallelepiped glass tube.

Acinetobacter sp. Tol5 cell suspension suspended in BS-N medium was flowed through the above flow cell at a rate of 64 μl/min for 30 minutes to attach Tol5 cell aggregates to the glass flow cell. Subsequently, the BS-N medium containing 0.1 mM AVL peptide, 0.1 mM ATL peptide or 0.1 mM SVL peptide was flowed at the same rate, and the behavior of the attached Tol5 cell agglomerates was continuously observed with a digital microscope (VHX-200 manufactured by KEYENCE Corporation). As a control, a BS-N medium free of the peptides was delivered at the same rate.

The composition of the BS-N medium used in Example 1 is described in the paper, (S. Ishii, J. Koki, H. Unno, K. Hori; Two Morphological Types of Cell Appendages on a Strongly Adhesive Bacterium, Acinetobacter sp. Strain Tol5, Appl. Environ. Microbiol. 70, (2004) 5026-5029) and the paper, (H. Watanabe, Y. Tanji, H. Unno, K. Hori; Rapid Conversion of Toluene by an Acinetobacter sp. Tol5 Mutant Showing Monolayer Adsorption to Water-Oil Interface, J. Biosci. Bioeng. 106 (2008) 226-230).

(Results and Discussion)

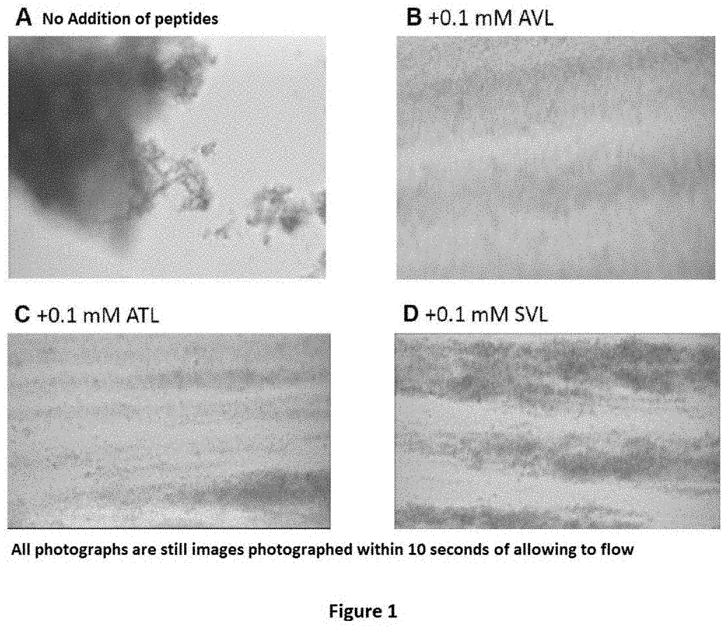

The results are shown in FIG. 1. As to the Acinetobacter sp. Tol5 strain, there was no change in the aggregates of the bacterial cells when the control, or the medium containing no peptides, was flowed, whereas when AVL peptide, ATL peptide or SVL peptide was added, rapid (within 10 seconds) disintegration of the bacterial cell aggregation of the Acinetobacter sp. Tol5 strain was observed. From this, it was confirmed that the AVL peptide, ATL peptide or SVL peptide is a factor that disintegrates the self-aggregation between AtaA proteins.

### Example 2: Separation Activity of AVL, ATL and SVL Peptides

This example shows that the binding between E. coli recombinant Nhead and streptavidin is separated by AVL, ATL and SVL peptides.

(Methods)

E. coli recombinant Nhead (fragment of AtaA polypeptide) was prepared according to the method described in (Manufacture Examples). An equal amount of 0.2 mg/ml of recombinant protein of Nhead (SEQ ID NO: 5) and 0.2 mg/ml of streptavidin were mixed and allowed to stand for 5 minutes. 40 μL of the above mixture was placed in a quartz cell, and the grain shape was measured with a DLS measuring device equipped with a He—Ne laser (wavelength 633 nm) (Zetasizer Nano ZSP, Malvern Instruments, UK), thus confirming that the recombinant protein of Nhead and streptavidin were bound to form a complex. Subsequently, AVL peptide was added to the mixture so as to reach 0.2 μg/ml, followed by mixing and allowing to stand for 5 minutes, and the DLS measurement was performed again. The DLS measurement was performed under the conditions of a temperature of 25° C., an equilibrium time of 120 seconds, and a scattering angle of 173°.

(Results and Discussion)

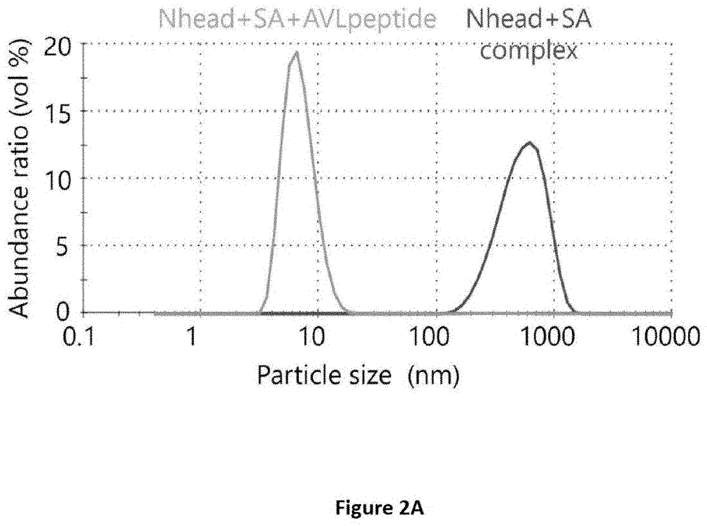

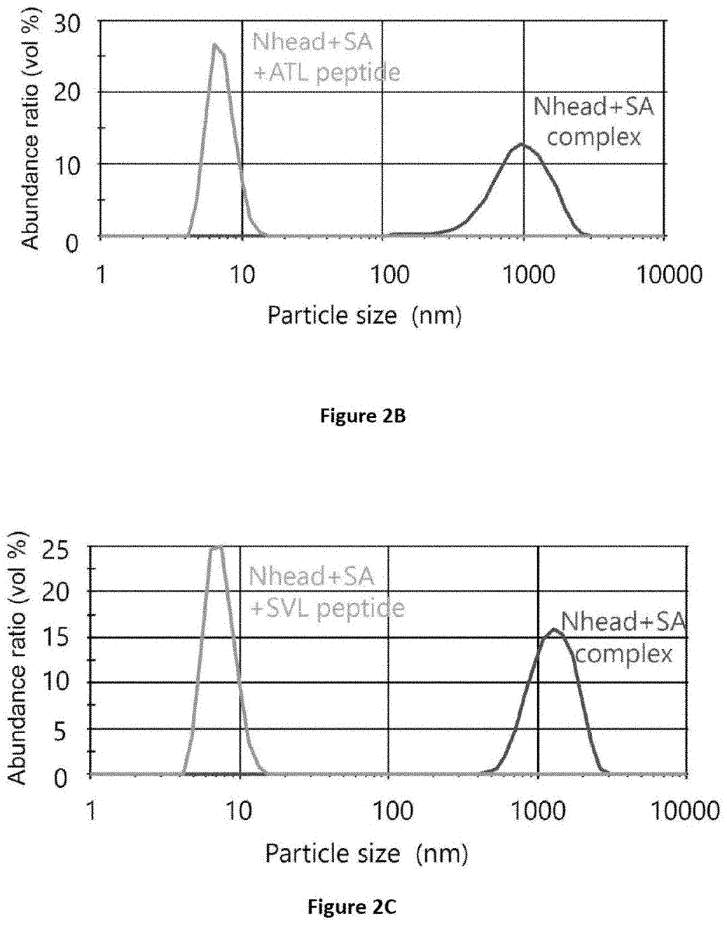

The results are shown in FIGS. 2A-2C. In the complex of Nhead and streptavidin, a diameter peak was observed at a particle size of about 1000 nm, whereas when AVL peptide (FIG. 2A), ATL peptide (FIG. 2B) and SVL peptide (FIG. 2C) were added to the complex of Nhead and streptavidin, a diameter peak was observed in the particle size of about 5 to 10 nm. From this result, it was considered that the binding between Nhead and streptavidin was separated by the addition of AVL peptide, ATL peptide and SVL peptide.

### Example 3: Repeated Adhesion/Separation Operations for Biotinylated Molecules

This example shows the reversible immobilization of biotinylated molecules using the binding between Nhead and streptavidin.

(Methods)

Three solutions were prepared by individually dissolving E. coli recombinant Nhead protein, streptavidin, and biotinylated fluorescent peptide (in which the N-terminal of the peptide GGSGGSK (SEQ ID NO: 6) was modified with fluorescein and the C-terminal was modified with biotin) in PBS buffer to a concentration of 1 μM.

First, 100 μl of PBS buffer was applied to a black 96-well polystyrene plate for fluorescence measurement. Subsequently, the operation (1) below was performed to measure the negative control value (blank fluorescence value due to streptavidin adsorbed on the plate) prior to the immobilizing of the Nhead protein. Next, 100 μl of Nhead protein solution was applied. The Nhead protein was adhered to the plate by allowing it to stand at 37° C. for 1 hour. Thereafter, the following operations were repeated in the order of (1)→(2)→(1)→(2)→(1).

(1) Streptavidin binding operation
(1-1). The solution was removed, and 100 μl of streptavidin solution was applied.
(1-2). Streptavidin was caused to bind to the Nhead protein immobilized on the plate by allowing it to stand at 28° C. for 30 minutes.
(1-3). The solution was removed and 100 μl of biotinylated fluorescent peptide was applied.
(1-4). The biotinylated fluorescent peptide was caused to bind to streptavidin by allowing it to stand at 28° C. for 30 minutes.
(1-5). The above was washed 5 times with 200 μl PBS buffer.
(1-6). A 100 μl PBS buffer was applied and fluorescence measurement (excitation wavelength: 485 nm, fluorescence wavelength: 535 nm) was performed with a plate reader (ARVO X3 manufactured by PerkinElmer).
(1-7). The buffer was removed.
(2) Streptavidin separation operation
(2-1). The above was washed twice with 200 μl of 100 μM AVL peptide solution and once with 200 μl of PBS buffer (applied and removed as is).
(2-2). Residual fluorescence was determined by applying 100 μl of PBS buffer and re-measuring the fluorescence value with a plate reader.

(Results and Discussion)

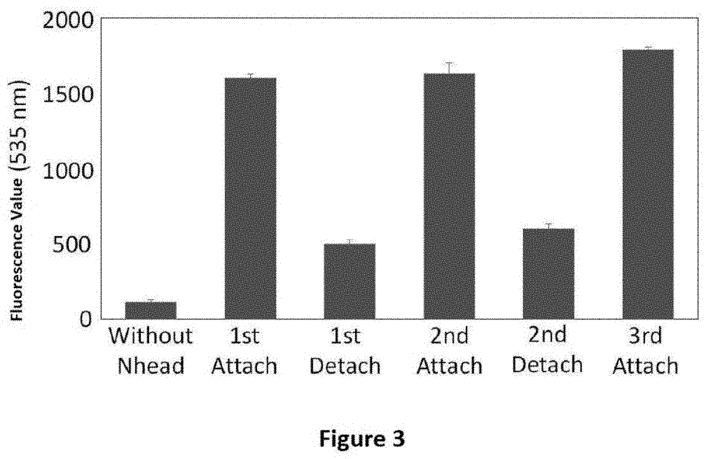

The results are shown in FIG. 3. In the fluorescence measurement performed after the first binding reaction of the biotinylated fluorescent peptide, an increase in the fluorescence value was observed. This result demonstrated that the biotinylated fluorescent peptide was bound to the complex of Nhead and streptavidin adhered to the polystyrene plate. In the fluorescence measurement performed after the addition of the AVL peptide, a decrease in the fluorescence value was observed, indicating that the binding between Nhead and streptavidin was separated. Similar results were observed after the second binding reaction and separation reaction, and after the third binding reaction, indicating that the binding between Nhead and streptavidin was reversibly bound and separated.

(Note)

As described above, the present disclosure is exemplified by the use of its preferred embodiments. However, the above descriptions and examples are provided for purposes of illustration only and not for the purpose of limiting the present disclosure. Accordingly, it is understood that the present invention is not limited to the embodiments or examples specifically described herein and the scope of the present disclosure should be interpreted solely based on the Claims. It is also understood that any patent, any patent application, and any references cited herein should be incorporated herein by reference in the same manner as the contents are specifically described herein. The present application claims priority to Japanese Patent Application No. 2019-132490 filed on Jul. 18, 2019 with the Japan Patent Office. It is understood that the entire content thereof is incorporated herein by reference.

### INDUSTRIAL APPLICABILITY

The present disclosure has utility in industries that use bioprocesses.

### [Sequence Listing Free Text]

SEQ ID NO: 1: amino acid sequence of AtaA fragment including methionine at the starting position, excluding the signal peptide
SEQ ID NO: 2: nucleic acid sequence of AtaA fragment including methionine at the starting position, excluding the signal peptide
SEQ ID NO: 3: full-length nucleic acid sequence of AtaA
SEQ ID NO: 4: full-length amino acid sequence of AtaA
SEQ ID NO: 5: amino acid sequence of recombinant protein in Nhead domain of AtaA
SEQ ID NO: 6: amino acid sequence of artificial peptide

1-11. (canceled)

12. A method for separating a bond that exists between a first subject and a second subject, comprising
contacting said first and second subjects with (i) a separated peptide which comprises a tripeptide of amino acid sequence alanine-valine-leucine (AVL), serine-valine-leucine (SVL), or alanine-threonine-leucine (ATL), said separated peptide having an N-terminus with 0, 1, 2, 3, 4, 5, 6, or 7 amino acids added to the N-terminus and a C-terminus with 0, 1, 2, 3, 4, 5, 6, or 7 amino acids added to the C-terminus, or (ii) a functional equivalent of the separated peptide of (i),
wherein the first subject comprises a polypeptide comprising the amino acid sequence set forth in SEQ ID NO: 1 or an altered sequence thereof or a fragment thereof, or a fusion or complex of the polypeptide with another amino acid sequence.

13. The method of claim 12, wherein the second subject comprises a functionality-impairing entity.

14. The method of claim 12, wherein the second subject comprises streptavidin, neutral avidin or a variant of either thereof.

15. The method of claim 13 wherein the functionality-impairing entity comprises an enzyme, antibody, fluorescent molecule, receptor, antibacterial peptide, signaling factor, inducing factor, nucleic acid aptamer, serum component, or a cell.

16. The method of claim 12 wherein the separated peptide comprises a tripeptide of amino acid sequence alanine-valine-leucine (AVL), serine-valine-leucine (SVL), or alanine-threonine-leucine (ATL) and has either one or both of (a) 0 amino acids added to the N-terminus of said tripeptide, and (b) 0 amino acids added to the C-terminus of said tripeptide.

17. The method of claim 12 wherein the separated peptide consists of a tripeptide of amino acid sequence alanine-valine-leucine (AVL), serine-valine-leucine (SVL), or alanine-threonine-leucine (ATL).

18. The method of claim 12 wherein the first subject comprises a polypeptide comprising the amino acid sequence set forth in SEQ ID NO: 1 or an altered sequence thereof or a fragment thereof.

19. The method of claim 12 wherein the first subject comprises a polypeptide comprising the amino acid sequence set forth in SEQ ID NO: 1.

20. The method of claim 12 wherein the first subject comprises a polypeptide comprising the amino acid sequence set forth in SEQ ID NO: 1 or an altered sequence thereof or a fragment thereof which comprises an amino acid sequence that is at least 30%, 40%, 50%, 60%, 70%, 80%, 90%, 95%, or 99% identical to SEQ ID NO: 1.


SEQUENCE LISTING (6 sequences)

SEQ ID NO: 1 - protein, 3518 aa, artificial sequence
  Fragment of AtaA
MATVQVGGGTNSGTTATASTNCADLYNYQNPENSGSGAAGNYNAGNPSVCSIAIGENAQGGTSGTGGSPGIAIGGNSKATGGLSVAIGGYAQATNVGSIALGTAALSSGFNSLAISRQAAATNNYSIAIGTTSVSKGVGSIAMGHSTNASGDQSIAIGSSDAVNSATATTTYDGTTNTQASGSKSIAIGASAKASTNNSIALGAGSVTSAQSGNSYLTGVGASATNGVVSVGTSTATRRIQNVADGSAASDAVTVAQLDKAYDDTNGRLAAALGTGSGAAYNAANNTYTAPTNIGGTGKNTIDDAIKATQRSVVAGSNIVVTPTTASDGSISYSVATSATPTFTSITVNNAPTAGTDATNKTYVDSKAAASRTEVAAGSNVSGVVKTTGANGQDVYTVNANGTTASAGSSAVTVTPGTKDANNVTDYKVDLSATTKTDIQKGVDAKNAVDTAGLKFKGDTATTSNTKKLGDTVSITGDTNISTVATTDGVQVKLNPNLDLGATGSVKTGNTTINNAGVTADQVTVGGVVINNTSGINAGGKAITNVAAPTNNTDAANKKYVDDAGTALTNLGFGLKAQDGTTVNKKLGEAVDIVGSNSNISTKVNAGKVEVALSNTLDLGTTGSVTTGSTVINNAGVTATQVTANKVTINNAPTAGTDATNKTYVDSKAAASRTEVAAGSNVSGVVKTTGANGQDIYAVNANGTTASAGSSAVTVTPGTKDANNVTDYKVDLSATTKTDIQKGVDAKNAVDTAGLKFKGDTATTSNTKKLGDTVSITGDTNISTVATTDGVQVKLNPNLDLGATGSVKTGNTTINNAGVTADQVTVGGVVINNTSGINAGGKAITNVAAPTNNTDAANKKYVDDAGTALTNLGFGLKAQDGTTVNKKLGEAVDIVGSNSNISTKVNAGKVEVALSNTLDLGTTGSVTTGSTVINNAGVTATQVTANKVTVNNAPTAGTDATNKTYVDSKAAASRTEVAAGSNVSGVVKTTGANGQDVYTVNANGTTASAGSSAVTVTPGTKDANNVTDYKVDLSATTKTDIQKGVDAKNAVDTAGLKFKGDTATTSNTKKLGDTVSITGDTNISTVATTDGVQVKLNPNLDLGATGSVKTGNTTINNAGVTADQVTVGGVVINNTSGINAGGKAITNVAAPTNNTDAANKKYVDDAGTALTNLGFGLKAQDGTTVNKKLGEAVEVVGADSNITTKVAGGQVAIELNKNLNNLTGITVNDGTNGTNGSTVIGKDGISVKDGSGNTIAGVDNTALTVKDGSGNTETSINQAINTLNAAQGETDKFAVKYDKNADGSVNYNNITLAGTTASSTQDATTGKITTTGGTSLNNVASAGDYKDVANASKGVNAGDLNNAVVDATNAATSKGFALQAADGAKVQKNLGEAVEVVGADSNITTKVAGGQVAIELNKNLNNLTGITVNDGTNGTNGSTVIGKDGISVKDGSGNTIAGVDNTALTVKDGSGNTETSINQAINTLNAAQGETDKFAVKYDKNTDGSTNYNSITAGNGNGTAATIGTDTAGNSVVTSGGTKISNVANGVNASDAVNKGQLDSLSTGLTNTGFGLKAADGNTVNKKLGEAVDVVGADSNITTKVAGGQVAIELNKNLNNLTGITVNDGTNGTNGSTVIGKDGISIKDGSGNTIAGVDNTALTVKDGSGNTETSINQAINTLNAAQGETDKFAVKYDKNADGSANYNNITLAGTTASSTQDATTGKITTTGGTSLNNVASAGDYKDVANASKGVNAGDLNNAVVDATNAATSKGFALQAADGAKVQKNLGEAVEVVGADSNITTKVVGGQVAIELNKNLNNLTGITVNDGTNGTNGSTVIGKDGISVKDGSGNTIAGVDNTALTVKDGSGNTETSINQAINTLNAAQGETDKFAVKYDKNADGSVNYNNITLAGTTASSTQDATTGKITTTGGTSLNNVASAGDYKDVANASKGVNAGDLNNAVVDATNAATSKGFALQAADGAKVQKNLGEAVEVVGADSNITTKVAGGQVAIELNKNLNNLTGITVNDGTNGTNGSTVIGKDGISVKDGSGNTIAGVDNTALTVKDGSGNTETSINQAINTLNAAQGETDKFAVKYDKNADGSVNYNNITLAGTTASSTQDATTGKITTTGGTSLNNVASAGDYKDVANASKGVNAGDLNNAVVDATNAATSKGFALQAADGAKVQKNLGEAVEVVGADSNITTKVAGGQVAIELNKNLNNLTGITVNDGTNGTNGSTVIGKDGISVKDGSGNTIAGVDNTALTVKDGSGNTETSINQAINTLNAAQGETDKFAVKYDKNADGSANYNNVTLAGTNGTIISNVKAGAVTSTSTDAINGSQLYGVANSVKNAIGGSTTIDATTGAITTTNIGGTGSNTIDGAISSIKDSATKAKTTVSAGDNVVVTSGTNADGSTNYEVATAKDVNFDKVTVGSVVVDKSSNTIKGLSNTTWNGTAVSGQAATEDQLKTVSDAQGETDKFAVKYDKNADGSANYNSITAGNGNGTAATIGTDTAGNSVVTSGGTKISNVANGVNASDAVNKGQLDSLSTGLTNTGFGLKAADGNTVNKKLGEAVDVVGADSNITTKVAGGQVAIELNKNLNNLTGITVNDGTNGTNGSTVIGKDGISIKDGSGNTIAGVDNTALTVKDSSGNTETSINQAINTLNAAQGETDKFAVKYDKNADGSVNYNNVTLAGTNGTIIRNVKAGAVTSTSTDAINGSQLYDIANSVKNAIGGSTTRDVTTGAITTTNIGGTGSNTIDGAISSIKDSATKAKTTISAGDNVVVTSGTNADGSTNYEVATAKDVNFDKVTVGNVVVDKANDTIQGLSNKDLNSTDFATKGRAATEEQLKAVITSNITEVVDGNGNKVNIIDQVVNTKPDNKNQDSLFLTYDKQGQETTDRLTIGQTVQKMNTDGIKFFHTNADTSKGDLGTTNDSSAGGLNSTAIGVNAIVANGADSSVALGHNTKVNGKQSIAIGSGAEALGNQSISIGTGNKVTGDHSGAIGDPTIVNGANSYSVGNNNQVLTDDTFVLGNNVTKTIAGSVVLGNGSAATTGAGEAGYALSVATNADKAAITKTTSSTGAVAVGDASSGIYRQITGVAAGSVDSDAVNVAQLKAVGNQVVTTQTTLVNSLGGNAKVNADGTITGPTYNVAQGNQTNVGDALTALDNAINTAATTSKSTVSNGQNIVVSKSKNADGSDNYEVSTAKDLTVDSVKAGDTVLNNAGITIGNNAVVLNNTGLTISGGPSVTLAGIDAGNKTIQNVANAVNATDAVNKGQLDSAINNVNNNVNELANNAVKYDDASKDKITLGGGATGTTITNVKDGTVAQGSKDAVNGGQLWNVQQQVDQNTTDISNIKNDINNGTVGLVQQAGKDAPVTVAKDTGGTTVNVAGTDGNRVVTGVKEGAVNATSKDAVNGSQLNTTNQAVVNYLGGGAGYDNITGSFTAPSYTVGDSKYNNVGGAIDALNQADQALNSKIDNVSNKLDNAFRITNNRIDDVEKKANAGIAAAMALESAPYVPG

SEQ ID NO: 2 - DNA, 10554 bp, artificial sequence
  Fragment of AtaA
atggctactgttcaagttggagggggaactaattctggaacaactgctacagcttctacgaattgtgcagacttatataattatcaaaatcctgagaactcaggctctggagcggctgggaattataatgcaggaaatccaagtgtgtgttcgatcgctataggtgaaaacgcacaaggtggtacttctggaactggagggtcgccagggatagcgataggtggaaattctaaagctacgggtggtttatctgttgctataggcggatatgctcaagcgacaaatgttggaagtattgctttaggcacagcagctttatcaagtggttttaacagtttagcaatatccagacaagctgctgcaacgaataactattcaatagctataggtacaacttcagtttcgaaaggagttggatcgattgctatggggcattcaacgaatgcttctggagatcaatcgatagcaattggtagctcggatgctgttaattcagcaacagcaacaacaacatacgatggtacaacaaatactcaagcatcaggtagtaaatcgattgctataggtgcaagcgcaaaggcatcaaccaataacagcattgcactaggtgcaggatcggtaacttctgcacaatctggtaattcttatcttactggtgtaggtgcatcagctacaaatggtgttgtatctgttggaacttcaactgcaacacgtcgtatccaaaatgtagcagatggttcagccgcttcagatgctgtgacagttgctcagttggataaagcttatgatgatacaaatggtcgtttagctgctgctttaggtacaggtagtggtgctgcctataatgcagcaaacaatacatataccgctccaacgaatattgggggaacaggtaaaaatacgattgatgatgcaattaaagcaactcaacgaagtgtagtcgctggatcaaatattgtcgttaccccgacgacagcttctgatggttcaatatcgtattcggttgctacaagcgcaacaccgacgtttacaagtataactgtaaacaatgcaccaacggcaggtacagatgcgaccaacaagacttatgtagactcaaaagcagcagcatcgagaacagaagtagcagctggaagcaatgtatctggtgtagtaaaaacgacaggcgcaaacggtcaagacgtttatacagtaaatgccaatggtacgactgcatcagcaggttcttcagcagttaccgtaacaccaggcacgaaagatgcaaataatgtcactgactataaagtagacttatcagcgactacaaaaaccgatatccaaaaaggtgtagatgcaaaaaatgctgtagataccgcaggtctaaaatttaaaggtgatacagcaaccacaagcaataccaagaaattaggtgacaccgtttcgattacgggtgatacgaacattagtacagttgcgacaacagatggtgtacaggttaagttaaatccaaacttggatttaggagcaactggtagcgttaaaacgggtaataccacgattaacaatgcaggtgtaacagctgatcaagttacggttggtggtgttgttattaacaacacatcaggtattaatgctggtggtaaagcgattactaatgtagcagcaccaacaaataacacagatgctgctaacaagaagtatgtagatgatgcaggtacagcattaaccaatttgggctttggattaaaagcacaagatggtacgactgtgaacaagaaattaggtgaagcagttgatattgttggttcaaacagcaacatcagtacaaaagtaaatgcaggcaaagtagaagttgcactatccaatacattggacttaggtactacaggtagcgttactacgggttcaactgtaattaacaatgctggtgttacggcaactcaagttaccgcaaacaaagtcacaataaacaatgcaccaacagcaggtacagatgcgaccaacaagacttatgtagactcaaaagcagcagcatcaagaacagaagtcgcagctggaagcaatgtatctggtgtagtaaaaacgacaggcgcaaacggtcaagatatttatgcagtaaatgccaatggtacgactgcatcagcaggttcttcagcagttaccgtaacaccaggcacgaaagatgcaaataatgtcactgactataaagtagacttgtcagcgactacaaaaaccgatattcaaaaaggtgtagatgcaaaaaatgctgtagatactgcaggtctaaaatttaaaggtgatacagcaaccacaagcaataccaagaaattaggtgacaccgtttcgattacgggtgatacgaacattagtacagttgcaacaactgatggtgtacaggttaagttaaatccaaacttagatttaggagcaactggtagcgttaaaacgggtaataccacgattaacaatgcaggtgtaacagctgaccaagttacggttggtggtgttgttattaacaacacatcaggtattaatgctggtggtaaagcgattaccaatgtagcagcaccaacaaataacacagatgctgctaacaagaagtatgtagatgacgcaggtacagcattaaccaatttgggctttggattaaaagcgcaagatggtacgactgtgaacaagaaattaggtgaagcagttgatattgttggttcaaacagcaacatcagtacaaaagtaaatgcaggcaaagtagaagttgcactatccaatacattggacttaggtactacaggtagcgttactacgggttcaactgtaattaacaatgctggtgttacggcaactcaagttaccgcaaacaaagtcacagttaataatgcaccaacagcaggtacagatgcgaccaataaaacttatgtagactcaaaagcagcggcatcaagaacagaagtcgcagctggaagcaatgtatctggcgtagtaaaaacgacaggtgcaaacggtcaagacgtttatacagtaaatgccaatggtacgactgcatcagcaggttcttcagcagttaccgtaacaccaggcacgaaagatgcaaataatgtcactgactataaagtagacttgtcagcgactacaaaaaccgatattcaaaaaggtgtagatgcaaaaaatgctgtagataccgcaggtctaaaatttaaaggtgatacagcaaccacaagcaataccaagaaattaggtgacaccgtttcgattacgggtgatacgaacattagtacagttgcgacaactgatggtgtacaggttaagctaaatccaaacttggatttaggagcaactggtagcgttaaaacgggtaataccacgattaacaatgcaggtgtaacagctgatcaagttacagttggtggtgttgttattaacaacacatcaggtattaatgctggtggtaaagcgattaccaatgtagcagcaccaacaaataacacagatgctgctaacaagaagtatgtagatgatgcaggtacagcattaaccaatttgggctttggattaaaagcgcaagatggtacgactgtgaacaagaaattaggcgaagcagttgaagttgttggtgcggacagtaacatcaccacgaaagttgcaggcggtcaggttgcaattgagttaaataaaaacctcaacaacttaactggcattaccgtgaacgatggaaccaatggcaccaatggttcaactgtgattggtaaagatggtatttcggttaaagatggttcaggcaataccattgcaggtgtagataacacagcgttgacagttaaagatggcagtggcaacacagaaaccagcattaaccaagcgatcaacacgttaaatgcagcgcaaggtgaaactgataagtttgcagtgaagtacgacaaaaatgctgatggcagtgtgaactacaacaacatcacattggcaggtacgactgcaagcagtacacaagatgcaactacaggcaagatcaccacaacaggtggaacaagcttgaacaatgttgcaagtgcgggtgactacaaagatgttgccaatgcaagcaaaggtgtaaacgcaggtgacttaaacaatgcagttgttgatgcaaccaatgcagcaaccagcaaaggctttgcattacaagcagcagatggcgctaaagttcagaagaacctaggcgaagcagttgaagttgtcggtgccgacagcaacatcaccacaaaagttgcaggcggtcaggttgcaattgagttaaataaaaacctcaacaacttaactggcattaccgtgaacgatggaaccaatggcaccaatggttcaactgtgattggtaaagatggtatttcagttaaagacggttcaggcaataccattgcaggtgtagataacacagcgttgacagttaaagatggcagtggcaacacagaaaccagcattaaccaagcgatcaacacgttaaatgcagcgcaaggtgaaactgataagtttgcagtgaagtacgacaaaaatacggatggtagtaccaactacaacagtattactgcaggcaatggtaacggtactgcagcaacgatcggaactgacacagcaggtaatagtgttgtgaccagtggcggaactaaaattagtaatgttgcgaatggtgtcaatgcaagtgatgcagtaaacaaaggtcaattggatagcttaagtacaggtcttaccaatacaggctttggtttaaaagcagcagatggcaacaccgttaacaaaaaattaggcgaagcagtagacgttgtcggtgctgacagcaacatcaccacgaaagttgcaggcggtcaggttgcgattgagttaaataaaaacctcaacaacttaactggcattaccgtgaacgatggaaccaatggcaccaatggttcaactgtgattggtaaagatggtatttcgattaaagatggttcaggcaataccattgcaggtgtagataacacagcgttgacagttaaagatggcagtggcaacacagaaaccagcattaaccaagcgatcaacacgttaaatgcagcgcaaggtgaaactgacaagtttgcagtgaagtacgacaagaatgctgatggcagtgcaaactacaacaacatcacattggcaggtacgactgcaagtagcacgcaagatgcaacaacaggcaagatcaccacaacaggtggaacaagcttgaacaacgttgcaagtgcaggtgactacaaagatgttgccaatgcaagcaaaggtgtaaacgcaggtgacttgaacaatgcagttgttgatgcaaccaatgcagcaaccagcaaaggctttgcattacaagcagcagatggcgctaaagttcagaagaacctaggcgaagcagttgaagttgtcggtgcggacagcaacatcaccacaaaagtagtgggtggacaagttgcgattgagttaaataaaaacctcaacaacttaactggcattaccgtgaacgatggaaccaatggcacaaatggttcaactgtgattggtaaagatggtatttcggttaaagatggttcaggtaataccattgcaggtgtagataacacagcgttgacagttaaagatggcagtggcaacacagaaaccagcattaaccaagcgatcaacacgttaaatgcagcgcaaggtgaaactgataagtttgcagtgaagtacgacaaaaatgctgatggcagtgtgaactacaacaacatcacattggcaggtacgactgcaagcagtacacaagatgcaactacaggcaagatcaccacaacaggtggaacaagcttgaacaatgttgcaagtgcgggtgactacaaagatgttgccaatgcaagcaaaggtgtaaacgcaggtgacttaaacaatgcagttgttgatgcaaccaatgcagcaaccagcaaaggctttgcattacaagcagcagatggcgctaaagttcagaagaacctaggcgaagcagttgaagttgtcggtgccgacagcaacatcaccacaaaagttgcaggcggtcaggttgcaattgagttaaataaaaacctcaacaacttaactggcattaccgtgaacgatggaaccaatggcaccaatggttcaactgtgattggtaaagatggtatttcagttaaagacggttcaggcaataccattgcaggtgtagataacacagcgttgacagttaaagatggcagtggcaacacagaaaccagcattaaccaagcgatcaacacgttaaatgcagcgcaaggtgaaactgataagtttgcagtgaagtacgacaaaaatgctgatggcagtgtgaactacaacaacatcacattggcaggtacgactgcaagcagtacacaagatgcaactacaggcaagatcaccacaacaggtggtacaagcttgaacaatgttgcaagtgcgggtgactacaaagatgttgccaatgcaagcaaaggtgtaaacgcaggtgacttgaacaatgcagttgttgatgcaaccaatgcagcgaccagcaaaggctttgcattacaagcagcagatggcgctaaagttcagaagaacctaggcgaagcagttgaagttgttggtgcggacagtaacatcaccacgaaagttgcaggcggtcaggttgcaattgagttaaataaaaacctcaacaacttaactggcattaccgtgaacgatggaaccaatggcaccaatggttcaactgtgattggtaaagatggtatttcggttaaagatggttcaggcaataccattgcaggtgtagataacacagcgttgacagttaaagatggcagtggcaacacagaaaccagcattaaccaagcgatcaacacgttaaatgcagcgcaaggtgaaactgataagtttgcagtgaagtacgacaaaaatgctgatggcagtgcaaactataacaatgtcactttagctggtacaaatggcacaataatcagcaatgttaaagcgggtgctgtgacctcaacatctactgatgcgatcaatggtagccaattatatggtgttgcaaacagcgtgaagaatgcaattggtggttcaaccacaattgatgcaacgactggtgcaatcacgacgaccaatattggtggtacaggttcaaatacgattgatggtgcaatcagcagtattaaagattcagcgactaaagcgaaaaccacggtaagtgctggggataatgttgtcgttacatcgggtaccaatgcagatggctcaacaaactatgaagttgcgacagcgaaagacgttaactttgacaaagtgactgtaggtagtgttgttgtagataaatcaagcaatacaatcaaaggattaagtaataccacttggaacggaacagcagtatcaggtcaagcggcgacagaagaccagttaaaaacggtcagcgatgcgcaaggtgaaactgataagtttgcagtgaagtacgacaaaaatgctgatggcagtgcgaactacaacagtattactgcaggcaatggtaacggtactgcagcaacgatcggaactgacacagcaggtaatagtgttgtgaccagtggcggaactaaaattagtaatgttgcgaatggtgtcaatgcaagtgatgcagtaaacaaaggtcaattggatagcttaagtacaggtcttaccaatacaggctttggtttaaaagcagcagatggcaacaccgttaacaaaaaattaggcgaagcagtagacgttgtcggtgctgacagcaacatcaccacgaaagttgcaggcggtcaggttgcgattgagttaaataaaaacctcaacaacttaactggcattaccgtgaacgatggaaccaatggcaccaatggttcaactgtgattggtaaagatggtatttcgattaaagatggttcaggcaataccattgcaggtgtagataacacagcgttgacggttaaagatagcagtggcaacacagaaaccagcattaaccaagcgatcaacacgttaaatgcagcgcaaggtgaaactgataagtttgcagtgaagtacgataagaatgctgatggcagtgtgaactataacaatgtcactttagcaggtacaaatggcacaataatcagaaatgttaaagcgggtgctgtgacctcaacatctactgatgcgatcaatggtagccaattatacgatattgcaaacagcgtgaagaatgcaattggtggttcaaccacaagagatgtaacgactggtgcaatcacaacgaccaatattggtggtacaggttcaaacacgattgatggtgcaatcagcagtattaaagattcagcgactaaagcgaaaaccacgataagtgctggggataatgttgtcgttacatcgggtaccaatgcagatggctcaacaaactatgaagttgcgacagcgaaagacgttaactttgacaaagtaactgtaggtaatgttgttgttgataaggcaaatgacacgatccaaggtttgagcaataaagatctaaattcaactgattttgcgaccaaaggtagagctgcgactgaagaacagttaaaagcagtgattaccagtaatatcacggaagttgtggatggtaatggcaacaaggtgaatattattgaccaagttgtaaataccaaacctgacaataagaaccaagattcattgttcttaacgtatgacaaacaaggtcaagaaaccacagatcgcctaacgattggtcaaacggtacagaagatgaatactgatggtattaaattcttccataccaatgccgatacatcaaaaggtgatttgggtacaacaaatgactcaagtgcaggtggtttaaactctacagcaattggtgtaaatgcgattgttgcgaatggtgcagatagttcagttgctttaggtcataacaccaaagtcaatggtaaacaatcaattgcaattggttctggtgcagaagctttaggcaatcaatcgatcagtattggtacaggcaataaagtcactggtgatcattcgggtgcgattggtgatccaactattgtaaatggtgcaaacagctactctgtgggtaataacaaccaagtacttacagatgacactttcgtacttggaaacaatgtcaccaaaactattgctggttcagtagtattgggtaacggttcagctgcaacgacaggtgctggtgaggcaggctatgccttatctgtagcaacaaatgcagataaagccgcgatcactaaaactacgtcaagcactggtgctgttgcagttggtgatgcgtcgagcggtatttatcgtcaaattaccggtgttgctgcgggtagcgtagattcagatgctgtgaacgttgcacagttaaaagcggtgggtaaccaagttgtaacgactcaaactacattggtgaacagtttgggtggtaacgctaaagtaaatgcagacggtacgattacaggaccaacttataatgttgctcaaggtaatcagaccaatgttggtgatgcattaactgcgcttgataacgcaattaatactgcggcaacaacatctaaatcgactgtttctaatggtcagaatattgttgtcagcaagagcaaaaatgcagatggttcagacaactatgaagtatcaacagcaaaagacttgacagttgattctgtcaaagcgggtgatacggttctgaataatgcaggtattacaattggcaataacgcagttgtattgaacaacactggattaaccattagtggtggaccaagtgttaccttggcaggcatcgatgcaggcaataaaaccattcaaaatgttgcgaatgcagtaaatgcaacagatgcagtcaacaaagggcaattggacagcgcaattaacaatgtgaataacaatgtaaatgagcttgccaacaacgctgttaaatatgacgatgcatcaaaagataagatcacacttggtggtggggcaactggtacaacaatcaccaatgtgaaagatggtactgttgcgcaaggttctaaagatgctgtgaatggcggtcaattgtggaatgttcaacaacaagttgatcagaacacaactgatattagcaatatcaaaaatgatattaacaacggtactgttggtttggttcaacaagcaggtaaagatgcaccagtgacggttgcaaaagatactggcggtacaacggtgaatgtcgctggaacagatggcaaccgagtagtgacaggtgttaaggaaggtgcagtgaatgcaacatctaaagatgctgtcaatggtagtcaattgaatacaaccaaccaagcggtagtcaattatcttggtggtggggcaggttatgacaacattacaggtagcttcacagcgccaagttatacggtaggtgactcgaaatacaacaatgttggtggcgcaattgatgcattgaatcaagcagatcaagcattgaatagcaaaattgacaatgtcagtaacaagttggataacgcattccgtattaccaacaaccgtattgatgatgtagagaaaaaagccaatgctggtattgccgctgcgatggctctggaatcagcaccatatgtcccaggt

SEQ ID NO: 3 - DNA, 10893 bp, Acinetobacter sp
  CDS (1)..(10893)
atgaataaaatctacaaagtgatttggaatgcgactttgttggcatgggttgcagtatctgaattggcaaaagggaaaaccaaatctacgacatcaaaatccaaagctaaatcattatcttcatctgtaatagttggtgggataatattaacaacacctttatctttaatagcagctactgttcaagttggagggggaactaattctggaacaactgctacagcttctacgaattgtgcagacttatataattatcaaaatcctgagaactcaggctctggagcggctgggaattataatgcaggaaatccaagtgtgtgttcgatcgctataggtgaaaacgcacaaggtggtacttctggaactggagggtcgccagggatagcgataggtggaaattctaaagctacgggtggtttatctgttgctataggcggatatgctcaagcgacaaatgttggaagtattgctttaggcacagcagctttatcaagtggttttaacagtttagcaatatccagacaagctgctgcaacgaataactattcaatagctataggtacaacttcagtttcgaaaggagttggatcgattgctatggggcattcaacgaatgcttctggagatcaatcgatagcaattggtagctcggatgctgttaattcagcaacagcaacaacaacatacgatggtacaacaaatactcaagcatcaggtagtaaatcgattgctataggtgcaagcgcaaaggcatcaaccaataacagcattgcactaggtgcaggatcggtaacttctgcacaatctggtaattcttatcttactggtgtaggtgcatcagctacaaatggtgttgtatctgttggaacttcaactgcaacacgtcgtatccaaaatgtagcagatggttcagccgcttcagatgctgtgacagttgctcagttggataaagcttatgatgatacaaatggtcgtttagctgctgctttaggtacaggtagtggtgctgcctataatgcagcaaacaatacatataccgctccaacgaatattgggggaacaggtaaaaatacgattgatgatgcaattaaagcaactcaacgaagtgtagtcgctggatcaaatattgtcgttaccccgacgacagcttctgatggttcaatatcgtattcggttgctacaagcgcaacaccgacgtttacaagtataactgtaaacaatgcaccaacggcaggtacagatgcgaccaacaagacttatgtagactcaaaagcagcagcatcgagaacagaagtagcagctggaagcaatgtatctggtgtagtaaaaacgacaggcgcaaacggtcaagacgtttatacagtaaatgccaatggtacgactgcatcagcaggttcttcagcagttaccgtaacaccaggcacgaaagatgcaaataatgtcactgactataaagtagacttatcagcgactacaaaaaccgatatccaaaaaggtgtagatgcaaaaaatgctgtagataccgcaggtctaaaatttaaaggtgatacagcaaccacaagcaataccaagaaattaggtgacaccgtttcgattacgggtgatacgaacattagtacagttgcgacaacagatggtgtacaggttaagttaaatccaaacttggatttaggagcaactggtagcgttaaaacgggtaataccacgattaacaatgcaggtgtaacagctgatcaagttacggttggtggtgttgttattaacaacacatcaggtattaatgctggtggtaaagcgattactaatgtagcagcaccaacaaataacacagatgctgctaacaagaagtatgtagatgatgcaggtacagcattaaccaatttgggctttggattaaaagcacaagatggtacgactgtgaacaagaaattaggtgaagcagttgatattgttggttcaaacagcaacatcagtacaaaagtaaatgcaggcaaagtagaagttgcactatccaatacattggacttaggtactacaggtagcgttactacgggttcaactgtaattaacaatgctggtgttacggcaactcaagttaccgcaaacaaagtcacaataaacaatgcaccaacagcaggtacagatgcgaccaacaagacttatgtagactcaaaagcagcagcatcaagaacagaagtcgcagctggaagcaatgtatctggtgtagtaaaaacgacaggcgcaaacggtcaagatatttatgcagtaaatgccaatggtacgactgcatcagcaggttcttcagcagttaccgtaacaccaggcacgaaagatgcaaataatgtcactgactataaagtagacttgtcagcgactacaaaaaccgatattcaaaaaggtgtagatgcaaaaaatgctgtagatactgcaggtctaaaatttaaaggtgatacagcaaccacaagcaataccaagaaattaggtgacaccgtttcgattacgggtgatacgaacattagtacagttgcaacaactgatggtgtacaggttaagttaaatccaaacttagatttaggagcaactggtagcgttaaaacgggtaataccacgattaacaatgcaggtgtaacagctgaccaagttacggttggtggtgttgttattaacaacacatcaggtattaatgctggtggtaaagcgattaccaatgtagcagcaccaacaaataacacagatgctgctaacaagaagtatgtagatgacgcaggtacagcattaaccaatttgggctttggattaaaagcgcaagatggtacgactgtgaacaagaaattaggtgaagcagttgatattgttggttcaaacagcaacatcagtacaaaagtaaatgcaggcaaagtagaagttgcactatccaatacattggacttaggtactacaggtagcgttactacgggttcaactgtaattaacaatgctggtgttacggcaactcaagttaccgcaaacaaagtcacagttaataatgcaccaacagcaggtacagatgcgaccaataaaacttatgtagactcaaaagcagcggcatcaagaacagaagtcgcagctggaagcaatgtatctggcgtagtaaaaacgacaggtgcaaacggtcaagacgtttatacagtaaatgccaatggtacgactgcatcagcaggttcttcagcagttaccgtaacaccaggcacgaaagatgcaaataatgtcactgactataaagtagacttgtcagcgactacaaaaaccgatattcaaaaaggtgtagatgcaaaaaatgctgtagataccgcaggtctaaaatttaaaggtgatacagcaaccacaagcaataccaagaaattaggtgacaccgtttcgattacgggtgatacgaacattagtacagttgcgacaactgatggtgtacaggttaagctaaatccaaacttggatttaggagcaactggtagcgttaaaacgggtaataccacgattaacaatgcaggtgtaacagctgatcaagttacagttggtggtgttgttattaacaacacatcaggtattaatgctggtggtaaagcgattaccaatgtagcagcaccaacaaataacacagatgctgctaacaagaagtatgtagatgatgcaggtacagcattaaccaatttgggctttggattaaaagcgcaagatggtacgactgtgaacaagaaattaggcgaagcagttgaagttgttggtgcggacagtaacatcaccacgaaagttgcaggcggtcaggttgcaattgagttaaataaaaacctcaacaacttaactggcattaccgtgaacgatggaaccaatggcaccaatggttcaactgtgattggtaaagatggtatttcggttaaagatggttcaggcaataccattgcaggtgtagataacacagcgttgacagttaaagatggcagtggcaacacagaaaccagcattaaccaagcgatcaacacgttaaatgcagcgcaaggtgaaactgataagtttgcagtgaagtacgacaaaaatgctgatggcagtgtgaactacaacaacatcacattggcaggtacgactgcaagcagtacacaagatgcaactacaggcaagatcaccacaacaggtggaacaagcttgaacaatgttgcaagtgcgggtgactacaaagatgttgccaatgcaagcaaaggtgtaaacgcaggtgacttaaacaatgcagttgttgatgcaaccaatgcagcaaccagcaaaggctttgcattacaagcagcagatggcgctaaagttcagaagaacctaggcgaagcagttgaagttgtcggtgccgacagcaacatcaccacaaaagttgcaggcggtcaggttgcaattgagttaaataaaaacctcaacaacttaactggcattaccgtgaacgatggaaccaatggcaccaatggttcaactgtgattggtaaagatggtatttcagttaaagacggttcaggcaataccattgcaggtgtagataacacagcgttgacagttaaagatggcagtggcaacacagaaaccagcattaaccaagcgatcaacacgttaaatgcagcgcaaggtgaaactgataagtttgcagtgaagtacgacaaaaatacggatggtagtaccaactacaacagtattactgcaggcaatggtaacggtactgcagcaacgatcggaactgacacagcaggtaatagtgttgtgaccagtggcggaactaaaattagtaatgttgcgaatggtgtcaatgcaagtgatgcagtaaacaaaggtcaattggatagcttaagtacaggtcttaccaatacaggctttggtttaaaagcagcagatggcaacaccgttaacaaaaaattaggcgaagcagtagacgttgtcggtgctgacagcaacatcaccacgaaagttgcaggcggtcaggttgcgattgagttaaataaaaacctcaacaacttaactggcattaccgtgaacgatggaaccaatggcaccaatggttcaactgtgattggtaaagatggtatttcgattaaagatggttcaggcaataccattgcaggtgtagataacacagcgttgacagttaaagatggcagtggcaacacagaaaccagcattaaccaagcgatcaacacgttaaatgcagcgcaaggtgaaactgacaagtttgcagtgaagtacgacaagaatgctgatggcagtgcaaactacaacaacatcacattggcaggtacgactgcaagtagcacgcaagatgcaacaacaggcaagatcaccacaacaggtggaacaagcttgaacaacgttgcaagtgcaggtgactacaaagatgttgccaatgcaagcaaaggtgtaaacgcaggtgacttgaacaatgcagttgttgatgcaaccaatgcagcaaccagcaaaggctttgcattacaagcagcagatggcgctaaagttcagaagaacctaggcgaagcagttgaagttgtcggtgcggacagcaacatcaccacaaaagtagtgggtggacaagttgcgattgagttaaataaaaacctcaacaacttaactggcattaccgtgaacgatggaaccaatggcacaaatggttcaactgtgattggtaaagatggtatttcggttaaagatggttcaggtaataccattgcaggtgtagataacacagcgttgacagttaaagatggcagtggcaacacagaaaccagcattaaccaagcgatcaacacgttaaatgcagcgcaaggtgaaactgataagtttgcagtgaagtacgacaaaaatgctgatggcagtgtgaactacaacaacatcacattggcaggtacgactgcaagcagtacacaagatgcaactacaggcaagatcaccacaacaggtggaacaagcttgaacaatgttgcaagtgcgggtgactacaaagatgttgccaatgcaagcaaaggtgtaaacgcaggtgacttaaacaatgcagttgttgatgcaaccaatgcagcaaccagcaaaggctttgcattacaagcagcagatggcgctaaagttcagaagaacctaggcgaagcagttgaagttgtcggtgccgacagcaacatcaccacaaaagttgcaggcggtcaggttgcaattgagttaaataaaaacctcaacaacttaactggcattaccgtgaacgatggaaccaatggcaccaatggttcaactgtgattggtaaagatggtatttcagttaaagacggttcaggcaataccattgcaggtgtagataacacagcgttgacagttaaagatggcagtggcaacacagaaaccagcattaaccaagcgatcaacacgttaaatgcagcgcaaggtgaaactgataagtttgcagtgaagtacgacaaaaatgctgatggcagtgtgaactacaacaacatcacattggcaggtacgactgcaagcagtacacaagatgcaactacaggcaagatcaccacaacaggtggtacaagcttgaacaatgttgcaagtgcgggtgactacaaagatgttgccaatgcaagcaaaggtgtaaacgcaggtgacttgaacaatgcagttgttgatgcaaccaatgcagcgaccagcaaaggctttgcattacaagcagcagatggcgctaaagttcagaagaacctaggcgaagcagttgaagttgttggtgcggacagtaacatcaccacgaaagttgcaggcggtcaggttgcaattgagttaaataaaaacctcaacaacttaactggcattaccgtgaacgatggaaccaatggcaccaatggttcaactgtgattggtaaagatggtatttcggttaaagatggttcaggcaataccattgcaggtgtagataacacagcgttgacagttaaagatggcagtggcaacacagaaaccagcattaaccaagcgatcaacacgttaaatgcagcgcaaggtgaaactgataagtttgcagtgaagtacgacaaaaatgctgatggcagtgcaaactataacaatgtcactttagctggtacaaatggcacaataatcagcaatgttaaagcgggtgctgtgacctcaacatctactgatgcgatcaatggtagccaattatatggtgttgcaaacagcgtgaagaatgcaattggtggttcaaccacaattgatgcaacgactggtgcaatcacgacgaccaatattggtggtacaggttcaaatacgattgatggtgcaatcagcagtattaaagattcagcgactaaagcgaaaaccacggtaagtgctggggataatgttgtcgttacatcgggtaccaatgcagatggctcaacaaactatgaagttgcgacagcgaaagacgttaactttgacaaagtgactgtaggtagtgttgttgtagataaatcaagcaatacaatcaaaggattaagtaataccacttggaacggaacagcagtatcaggtcaagcggcgacagaagaccagttaaaaacggtcagcgatgcgcaaggtgaaactgataagtttgcagtgaagtacgacaaaaatgctgatggcagtgcgaactacaacagtattactgcaggcaatggtaacggtactgcagcaacgatcggaactgacacagcaggtaatagtgttgtgaccagtggcggaactaaaattagtaatgttgcgaatggtgtcaatgcaagtgatgcagtaaacaaaggtcaattggatagcttaagtacaggtcttaccaatacaggctttggtttaaaagcagcagatggcaacaccgttaacaaaaaattaggcgaagcagtagacgttgtcggtgctgacagcaacatcaccacgaaagttgcaggcggtcaggttgcgattgagttaaataaaaacctcaacaacttaactggcattaccgtgaacgatggaaccaatggcaccaatggttcaactgtgattggtaaagatggtatttcgattaaagatggttcaggcaataccattgcaggtgtagataacacagcgttgacggttaaagatagcagtggcaacacagaaaccagcattaaccaagcgatcaacacgttaaatgcagcgcaaggtgaaactgataagtttgcagtgaagtacgataagaatgctgatggcagtgtgaactataacaatgtcactttagcaggtacaaatggcacaataatcagaaatgttaaagcgggtgctgtgacctcaacatctactgatgcgatcaatggtagccaattatacgatattgcaaacagcgtgaagaatgcaattggtggttcaaccacaagagatgtaacgactggtgcaatcacaacgaccaatattggtggtacaggttcaaacacgattgatggtgcaatcagcagtattaaagattcagcgactaaagcgaaaaccacgataagtgctggggataatgttgtcgttacatcgggtaccaatgcagatggctcaacaaactatgaagttgcgacagcgaaagacgttaactttgacaaagtaactgtaggtaatgttgttgttgataaggcaaatgacacgatccaaggtttgagcaataaagatctaaattcaactgattttgcgaccaaaggtagagctgcgactgaagaacagttaaaagcagtgattaccagtaatatcacggaagttgtggatggtaatggcaacaaggtgaatattattgaccaagttgtaaataccaaacctgacaataagaaccaagattcattgttcttaacgtatgacaaacaaggtcaagaaaccacagatcgcctaacgattggtcaaacggtacagaagatgaatactgatggtattaaattcttccataccaatgccgatacatcaaaaggtgatttgggtacaacaaatgactcaagtgcaggtggtttaaactctacagcaattggtgtaaatgcgattgttgcgaatggtgcagatagttcagttgctttaggtcataacaccaaagtcaatggtaaacaatcaattgcaattggttctggtgcagaagctttaggcaatcaatcgatcagtattggtacaggcaataaagtcactggtgatcattcgggtgcgattggtgatccaactattgtaaatggtgcaaacagctactctgtgggtaataacaaccaagtacttacagatgacactttcgtacttggaaacaatgtcaccaaaactattgctggttcagtagtattgggtaacggttcagctgcaacgacaggtgctggtgaggcaggctatgccttatctgtagcaacaaatgcagataaagccgcgatcactaaaactacgtcaagcactggtgctgttgcagttggtgatgcgtcgagcggtatttatcgtcaaattaccggtgttgctgcgggtagcgtagattcagatgctgtgaacgttgcacagttaaaagcggtgggtaaccaagttgtaacgactcaaactacattggtgaacagtttgggtggtaacgctaaagtaaatgcagacggtacgattacaggaccaacttataatgttgctcaaggtaatcagaccaatgttggtgatgcattaactgcgcttgataacgcaattaatactgcggcaacaacatctaaatcgactgtttctaatggtcagaatattgttgtcagcaagagcaaaaatgcagatggttcagacaactatgaagtatcaacagcaaaagacttgacagttgattctgtcaaagcgggtgatacggttctgaataatgcaggtattacaattggcaataacgcagttgtattgaacaacactggattaaccattagtggtggaccaagtgttaccttggcaggcatcgatgcaggcaataaaaccattcaaaatgttgcgaatgcagtaaatgcaacagatgcagtcaacaaagggcaattggacagcgcaattaacaatgtgaataacaatgtaaatgagcttgccaacaacgctgttaaatatgacgatgcatcaaaagataagatcacacttggtggtggggcaactggtacaacaatcaccaatgtgaaagatggtactgttgcgcaaggttctaaagatgctgtgaatggcggtcaattgtggaatgttcaacaacaagttgatcagaacacaactgatattagcaatatcaaaaatgatattaacaacggtactgttggtttggttcaacaagcaggtaaagatgcaccagtgacggttgcaaaagatactggcggtacaacggtgaatgtcgctggaacagatggcaaccgagtagtgacaggtgttaaggaaggtgcagtgaatgcaacatctaaagatgctgtcaatggtagtcaattgaatacaaccaaccaagcggtagtcaattatcttggtggtggggcaggttatgacaacattacaggtagcttcacagcgccaagttatacggtaggtgactcgaaatacaacaatgttggtggcgcaattgatgcattgaatcaagcagatcaagcattgaatagcaaaattgacaatgtcagtaacaagttggataacgcattccgtattaccaacaaccgtattgatgatgtagagaaaaaagccaatgctggtattgccgctgcgatggctctggaatcagcaccatatgtcccaggtaaatatacctatgcagcaggcgcagcttaccacggtggtgaaaatgcggtaggtgtgactttacgtaaaactgcagacaatggtcgttggtcgattacaggcggtgtagctgcagcgtctcaaggcgatgcaagtgttcgtatcggtatcagcggtgtgattgactaa

SEQ ID NO: 4 - protein, 3630 aa, Acinetobacter sp
MNKIYKVIWNATLLAWVAVSELAKGKTKSTTSKSKAKSLSSSVIVGGIILTTPLSLIAATVQVGGGTNSGTTATASTNCADLYNYQNPENSGSGAAGNYNAGNPSVCSIAIGENAQGGTSGTGGSPGIAIGGNSKATGGLSVAIGGYAQATNVGSIALGTAALSSGFNSLAISRQAAATNNYSIAIGTTSVSKGVGSIAMGHSTNASGDQSIAIGSSDAVNSATATTTYDGTTNTQASGSKSIAIGASAKASTNNSIALGAGSVTSAQSGNSYLTGVGASATNGVVSVGTSTATRRIQNVADGSAASDAVTVAQLDKAYDDTNGRLAAALGTGSGAAYNAANNTYTAPTNIGGTGKNTIDDAIKATQRSVVAGSNIVVTPTTASDGSISYSVATSATPTFTSITVNNAPTAGTDATNKTYVDSKAAASRTEVAAGSNVSGVVKTTGANGQDVYTVNANGTTASAGSSAVTVTPGTKDANNVTDYKVDLSATTKTDIQKGVDAKNAVDTAGLKFKGDTATTSNTKKLGDTVSITGDTNISTVATTDGVQVKLNPNLDLGATGSVKTGNTTINNAGVTADQVTVGGVVINNTSGINAGGKAITNVAAPTNNTDAANKKYVDDAGTALTNLGFGLKAQDGTTVNKKLGEAVDIVGSNSNISTKVNAGKVEVALSNTLDLGTTGSVTTGSTVINNAGVTATQVTANKVTINNAPTAGTDATNKTYVDSKAAASRTEVAAGSNVSGVVKTTGANGQDIYAVNANGTTASAGSSAVTVTPGTKDANNVTDYKVDLSATTKTDIQKGVDAKNAVDTAGLKFKGDTATTSNTKKLGDTVSITGDTNISTVATTDGVQVKLNPNLDLGATGSVKTGNTTINNAGVTADQVTVGGVVINNTSGINAGGKAITNVAAPTNNTDAANKKYVDDAGTALTNLGFGLKAQDGTTVNKKLGEAVDIVGSNSNISTKVNAGKVEVALSNTLDLGTTGSVTTGSTVINNAGVTATQVTANKVTVNNAPTAGTDATNKTYVDSKAAASRTEVAAGSNVSGVVKTTGANGQDVYTVNANGTTASAGSSAVTVTPGTKDANNVTDYKVDLSATTKTDIQKGVDAKNAVDTAGLKFKGDTATTSNTKKLGDTVSITGDTNISTVATTDGVQVKLNPNLDLGATGSVKTGNTTINNAGVTADQVTVGGVVINNTSGINAGGKAITNVAAPTNNTDAANKKYVDDAGTALTNLGFGLKAQDGTTVNKKLGEAVEVVGADSNITTKVAGGQVAIELNKNLNNLTGITVNDGTNGTNGSTVIGKDGISVKDGSGNTIAGVDNTALTVKDGSGNTETSINQAINTLNAAQGETDKFAVKYDKNADGSVNYNNITLAGTTASSTQDATTGKITTTGGTSLNNVASAGDYKDVANASKGVNAGDLNNAVVDATNAATSKGFALQAADGAKVQKNLGEAVEVVGADSNITTKVAGGQVAIELNKNLNNLTGITVNDGTNGTNGSTVIGKDGISVKDGSGNTIAGVDNTALTVKDGSGNTETSINQAINTLNAAQGETDKFAVKYDKNTDGSTNYNSITAGNGNGTAATIGTDTAGNSVVTSGGTKISNVANGVNASDAVNKGQLDSLSTGLTNTGFGLKAADGNTVNKKLGEAVDVVGADSNITTKVAGGQVAIELNKNLNNLTGITVNDGTNGTNGSTVIGKDGISIKDGSGNTIAGVDNTALTVKDGSGNTETSINQAINTLNAAQGETDKFAVKYDKNADGSANYNNITLAGTTASSTQDATTGKITTTGGTSLNNVASAGDYKDVANASKGVNAGDLNNAVVDATNAATSKGFALQAADGAKVQKNLGEAVEVVGADSNITTKVVGGQVAIELNKNLNNLTGITVNDGTNGTNGSTVIGKDGISVKDGSGNTIAGVDNTALTVKDGSGNTETSINQAINTLNAAQGETDKFAVKYDKNADGSVNYNNITLAGTTASSTQDATTGKITTTGGTSLNNVASAGDYKDVANASKGVNAGDLNNAVVDATNAATSKGFALQAADGAKVQKNLGEAVEVVGADSNITTKVAGGQVAIELNKNLNNLTGITVNDGTNGTNGSTVIGKDGISVKDGSGNTIAGVDNTALTVKDGSGNTETSINQAINTLNAAQGETDKFAVKYDKNADGSVNYNNITLAGTTASSTQDATTGKITTTGGTSLNNVASAGDYKDVANASKGVNAGDLNNAVVDATNAATSKGFALQAADGAKVQKNLGEAVEVVGADSNITTKVAGGQVAIELNKNLNNLTGITVNDGTNGTNGSTVIGKDGISVKDGSGNTIAGVDNTALTVKDGSGNTETSINQAINTLNAAQGETDKFAVKYDKNADGSANYNNVTLAGTNGTIISNVKAGAVTSTSTDAINGSQLYGVANSVKNAIGGSTTIDATTGAITTTNIGGTGSNTIDGAISSIKDSATKAKTTVSAGDNVVVTSGTNADGSTNYEVATAKDVNFDKVTVGSVVVDKSSNTIKGLSNTTWNGTAVSGQAATEDQLKTVSDAQGETDKFAVKYDKNADGSANYNSITAGNGNGTAATIGTDTAGNSVVTSGGTKISNVANGVNASDAVNKGQLDSLSTGLTNTGFGLKAADGNTVNKKLGEAVDVVGADSNITTKVAGGQVAIELNKNLNNLTGITVNDGTNGTNGSTVIGKDGISIKDGSGNTIAGVDNTALTVKDSSGNTETSINQAINTLNAAQGETDKFAVKYDKNADGSVNYNNVTLAGTNGTIIRNVKAGAVTSTSTDAINGSQLYDIANSVKNAIGGSTTRDVTTGAITTTNIGGTGSNTIDGAISSIKDSATKAKTTISAGDNVVVTSGTNADGSTNYEVATAKDVNFDKVTVGNVVVDKANDTIQGLSNKDLNSTDFATKGRAATEEQLKAVITSNITEVVDGNGNKVNIIDQVVNTKPDNKNQDSLFLTYDKQGQETTDRLTIGQTVQKMNTDGIKFFHTNADTSKGDLGTTNDSSAGGLNSTAIGVNAIVANGADSSVALGHNTKVNGKQSIAIGSGAEALGNQSISIGTGNKVTGDHSGAIGDPTIVNGANSYSVGNNNQVLTDDTFVLGNNVTKTIAGSVVLGNGSAATTGAGEAGYALSVATNADKAAITKTTSSTGAVAVGDASSGIYRQITGVAAGSVDSDAVNVAQLKAVGNQVVTTQTTLVNSLGGNAKVNADGTITGPTYNVAQGNQTNVGDALTALDNAINTAATTSKSTVSNGQNIVVSKSKNADGSDNYEVSTAKDLTVDSVKAGDTVLNNAGITIGNNAVVLNNTGLTISGGPSVTLAGIDAGNKTIQNVANAVNATDAVNKGQLDSAINNVNNNVNELANNAVKYDDASKDKITLGGGATGTTITNVKDGTVAQGSKDAVNGGQLWNVQQQVDQNTTDISNIKNDINNGTVGLVQQAGKDAPVTVAKDTGGTTVNVAGTDGNRVVTGVKEGAVNATSKDAVNGSQLNTTNQAVVNYLGGGAGYDNITGSFTAPSYTVGDSKYNNVGGAIDALNQADQALNSKIDNVSNKLDNAFRITNNRIDDVEKKANAGIAAAMALESAPYVPGKYTYAAGAAYHGGENAVGVTLRKTADNGRWSITGGVAAASQGDASVRIGISGVID

SEQ ID NO: 5 - protein, 305 aa, artificial sequence
  Nhead-GCN4pII-His
MATVQVGGGTNSGTTATASTNCADLYNYQNPENSGSGAAGNYNAGNPSVCSIAIGENAQGGTSGTGGSPGIAIGGNSKATGGLSVAIGGYAQATNVGSIALGTAALSSGFNSLAISRQAAATNNYSIAIGTTSVSKGVGSIAMGHSTNASGDQSIAIGSSDAVNSATATTTYDGTTNTQASGSKSIAIGASAKASTNNSIALGAGSVTSAQSGNSYLTGVGASATNGVVSVGTSTATRRIQNVADGSAASDAVTVAQLDKAYDDTNGRMKQIEDKIEEILSKIYHIENEIARIKKLIKLHHHHHH

SEQ ID NO: 6 - protein, 7 aa, artificial sequence
  Synthetic Peptide
GGSGGSK